(19) United States
(12) Patent Application Publication
(10) Pub. No.: US 2009/0312295 A1
(43) Pub. Date: Dec. 17, 2009

(54) Second line treatment of metastatic hormone refractory prostate cancer using satraplatin

(76) Inventors: Thomas J. McKearn, New Hope, PA (US); Marcel Rozencweig, Princeton, NJ (US)

Correspondence Address: LEON R. YANKWICH, 201 BROADWAY, CAMBRIDGE, MA, 02139, US

(21) Appl. No.: 12/311,032

(22) PCT Filed: Sep. 24, 2007
(86) PCT No.: PCT/EP2007/060112

§ 371 Date: Aug. 25, 2009

Related U.S. Application Data
(60) Provisional application No. 60/846,968, filed on Sep. 24, 2006.
(60) Provisional application No. 60/903,008, filed on Feb. 22, 2007.
(60) Provisional application No. 60/903,180, filed on Feb. 23, 2007.

(30) Foreign Application Priority Data
Aug. 22, 2007 (EP) 07114786.2

(51) Int. Cl. A61K 31/282; A61K 31/573; A61K 31/439; A61P 35/00

(52) U.S. Cl. 514/171; 514/492; 514/299

(57) ABSTRACT

The instant invention relates to methods using satraplatin, packaged-pharmaceutical-products that include satraplatin and uses of satraplatin to prepare pharmaceutical compositions for the 2^{nd} line treatment of metastatic hormone refractory prostate cancer.

### BACKGROUND TO THE INVENTION

### Prostate Cancer

Worldwide, prostate cancer ranks as the second most common cancer in males, after lung cancer, and in the United States (U.S.) prostate cancer is the second leading cause of death from cancer in men. There were over 180,000 new cases and 29,000 deaths reported in the U.S. in the year 2002 (American Cancer Society). The frequency of patients presenting at each stage of disease has changed remarkably with introduction of prostate specific antigen (PSA) screening in the early 1990s.

Approximately 30-35% of patients with prostate cancer will present with regional or metastatic tumors, while an additional 25% will develop metastases in the course of the disease. Metastases are commonly to the bone, where the lesions can be seen on X-ray as osteosclerotic lesions or on a bone scan as areas of increased activity or “hot spots.” In patients presenting with metastatic disease and receiving androgen ablation, median survival is 2.5 years (Sternberg, 1992). In many such patients, bone pain and decreased performance status are predominant. Relief from these symptoms is as important as prolongation of survival. As a result, assessment of these symptoms, including pain, has become a fundamental part of many prostate cancer studies.

Patients with metastatic disease are initially treated with hormone therapy such as luteinizing hormone releasing hormone (LHRH) agonists, diethylstilbestrol (DES), orchiectomy, and/or anti-androgens. The development of hormonal resistance occurs in most patients after androgen deprivation. The term “hormone-refractory prostate cancer” (HRPC) is used by physicians to describe prostate cancer disease that progresses despite castrate levels of serum testosterone.

The median time to progression to HRPC is 18 months from the time of initiation of hormonal therapy against prostate cancer. Responses to current second line hormonal therapies are temporary and do not impact upon survival. The median survival after developing HRPC has been 12 to 18 months, and until recently, there was no clearly effective systemic treatment for this condition. With recent advances in the understanding of HRPC, novel treatment regimens are being identified. In the past, all treatments involving cytotoxic chemotherapy were considered inactive, but newer chemotherapy drugs and drug combinations are now demonstrating improved response rates (Kelly, 2000) and improved survival (Petrylak, 2004; Eisenberger, 2004).

Evaluation of tumor response in advanced prostate cancer has been difficult due to the predominance of non-measurable bony metastases and the infrequent presence of measurable lesions. More recently, PSA level has been used to evaluate the disease status of patients with prostate cancer.

PSA level is generally considered to be a useful surrogate measure in patients receiving hormonal therapy (Bubley, 1999; Miller, 1992), and it may be useful as well in patients receiving therapy for hormone refractory disease (Bubley, 1999; Kelly, 1993). There may be limitations for use of PSA levels to monitor disease in this population, however, since any new therapy may modulate PSA production by tumor cells independently of its effect or lack of effect on tumor growth (Eisenberger, 1996).

### Satraplatin

Satraplatin (INN/USAN), also known as JM-216, or bis(acetato) ammine dichloro (cyclohexylamine) platinum (IV), is a member of a novel class of platinum (IV) compounds that are absorbed by the oral route. The lipophilic properties of these compounds, and hence their absorption, are largely determined by the nature of the axial acetate ligands. Unlike the square planar platinum (II) complexes cisplatin and carboplatin, satraplatin is an octahedral platinum (IV) compound.

The molecular formula for satraplatin is C₁₀H₂₂N₂Cl₂O₄Pt. Its molecular weight is 500.29. Its chemical structure is:

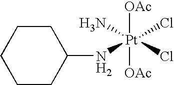

Satraplatin can be synthesised according to the method disclosed in U.S. Pat. Nos. 5,072,011 and 5,244,919 or by appropriate modification of the method disclosed in U.S. Pat. No. 6,518,428.

Upon administration of satraplatin to a cell, animal or a human patient, a number of metabolites may be formed. FIG. 1 (taken from Raynaud et al. 1996 Cancer Chemother. Phamacol. 38: 155-162) shows exemplary metabolites of satraplatin (JM216), and depicts JM118, JM383, JM518, JM559 and JM149.

### Differences Between Satraplatin and Other Platinum-Containing Therapeutic Agents

Satraplatin is a third-generation platinum compound studied in a variety of tumors. Since their original discovery, platinum compounds (cisplatin, carboplatin, oxaliplatin) have emerged as important agents for the therapy of several human tumors including testicular, bladder, lung, colorectal, head and neck, ovarian, and cervical cancer (Rozencweig, 1977; Loehrer, 2984; Prestayko, 1979). Cisplatin, used as single agent, has been evaluated in several trials for the treatment of hormone refractory carcinoma of the prostate (e.g. Rossof, 1979; Merrin, 1979; Yagoda, 1979(I); Yagoda, 1979(II); Qazi, 1983; Soloway, 1983; Moore, 1986). The primary endpoints in these studies were response rate in measurable disease. The response rates to single agent cisplatin are generally low or poor (see below) and comparable to those seen with other agents in this disease (Rossof, 1979; Yagoda, 1993). Furthermore, cisplatin was repeatedly shown not to be effective against prostate cancer. Qazi & Khandekar (Am. J. Clin. Oncol. (1983) 6, 203) demonstrated in a phase II trial that cisplatin is not effective in patients with metastatic prostatic carcinoma. Hasegawa et al. (Cancer & Chemother. (1987) 14, 3279) reported that the range of effective dose was wider for other platinum agents like carboplatin than for cisplatin. Even in combination treatment, cisplatin-comprising regimens demonstrate limited activity, e.g. in combination with mitoxantrone in metastatic prostate cancer (Osborne et al., Eur. J. Cancer (1992) 28, 477). Therefore, cisplatin has not been established as compound for chemotherapy of prostate cancer.

Although (i) pre-clinical studies with satraplatin demonstrated cytotoxic and anti-tumor activities comparable to cisplatin or carboplatin, and (ii) early clinical studies demonstrated its activity against platinum-sensitive tumors of the ovary and lung in addition to the prostate; satraplatin shows considerable and significant differences to other platinum agents, like e.g. cisplatin. It has shown activity in some platinum resistant tumor models in vitro, and unlike other platinum compounds, it is absorbed when administered orally. Using a panel of ovarian cancer carcinoma cell lines, Kelland et al. (Cancer Res (1992), 52, 822) demonstrated that satraplatin is significantly more cytotoxic than cisplatin, and that satraplatin exhibits selective cytotoxic effects against intrinsically cisplatin-resistant cell lines. Loh et al. (Br. J. Cancer (1992) 66, 1109) confirmed these findings. Loh et al. furthermore came to the conclusion that the increased accumulation of satraplatin, which is a result of its enhanced lipophilicity, accounts for the dramatic increase of the potency of satraplatin over cisplatin. Other studies reporting on the activity of satraplatin towards cell lines with acquired or intrinsic resistance to cisplatin are those of Mellish et al. (Br J Cancer (1993) 68, 240), using human cervical squamous cell carcinoma cell lines, and Orr et al. (Br J Cancer (1994) 70, 415), using murine leukemia cell lines. In the latter report the cell lines used were not just resistant to cisplatin, but also to tetraplatin and carboplatin.

Twentyman et al. (Cancer Res (1992) 52, 5674) investigated the sensitivity of human lung cancer cell lines with acquired or inherent resistance to cisplatin, to a series of novel platinum compounds, including satraplatin. In this study, cisplatin and carboplatin were found to act very similar, whereas satraplatin did not.

In spite of different routes of administration Kelland et al. (Int. J. Oncol. (1993) 2, 1043) demonstrated the surprising finding that the efficacy of orally administered satraplatin is comparable to that of cisplatin and carboplatin administered intravenously, as determined in human ovarian carcinoma xenograft models. These findings were confirmed by Rose et al. (Cancer Chemother. Pharmacol. (1993) 32, 197), using murine and human tumor models. McKeage et al. (Cancer Res. (1994) 54, 4118) investigated the differences of the schedule dependencies associated with these routes of administration.

In another study by Kelland et al. (Cancer Res. (1993) 53, 2581) many of the above mentioned differences between satraplatin and cisplatin were confirmed. Furthermore it was found, that the cytotoxicity of satraplatin was dependent on the time of drug exposure. Again, it was confirmed that satraplatin does not exhibit cross resistance to cisplatin, whereas other platinum agents, e.g. tetraplatin, do. Without being bound to any particular theory, satraplatin circumvents transport-determined acquired resistance to cisplatin.

Mellish et al. (Cancer Res. (1994) 54, 6194) investigated the mechanisms of acquired resistance to satraplatin in two human ovarian carcinoma cell lines. They found that, in contrast to cisplatin, acquired resistance to satraplatin is not mediated through reduced drug accumulation, but by increased intracellular GSH levels or increased DNA repair.

Sharp et al. (Clin. Cancer Res. 1995, 1, 981) compared the transport of cisplatin and satraplatin in human ovarian carcinoma cell lines. Cisplatin transport in the parental cell lines occurs via passive diffusion and active/facilitated transport, whereas in a cisplatin-resistant cell lines cisplatin enters cells by passive diffusion only. Without being bound to any particular theory, satraplatin circumvents cisplatin resistance by increasing the drug uptake. The mechanism of satraplatin transport across cell membranes is through passive diffusion, predominantly as a result of its enhanced lipophilicity.

Fink et al. (Cancer Res (1996) 56, 4881) investigated the effect of the loss of DNA mismatch repair activity on the sensitivity to cisplatin, satraplatin and other platinum agents. In contrast to cisplatin and carboplatin, which form the same type of adducts in DNA, there was no difference in sensitivity between mismatch repair-proficient and mismatch repair-deficient cell lines for satraplatin.

Perego et al. (Mol. Pharmacol. 1998, 54, 213) investigated the sensitivity of strains of Schizosaccharomyces pombe to cisplatin, satraplatin and other platinum compounds. The panel of the 23 yeast strains tested comprised many mutants in genes that affect the response to radiation. Whereas the mutants fell into three groups with respect to their sensitivity to cisplatin (minimal change in sensitivity, hypersensitivity, and marked hypersensitivity), none of the mutants demonstrated an appreciable change in sensitivity to satraplatin.

Leyland-Jones et al. (Amer. J. Pathol. 1999, 155, 77) investigated genomic imbalances associated with acquired resistance to platinum analogues. Using three ovarian carcinoma cell lines they identified differences between the three platinum compounds cisplatin, satraplatin and AMD473 (picoplatin).

Amorino et al. (Int. J. Radiation Oncol. Biol. Phys. 1999, 44, 399) investigated radiopotentiation by satraplatin and the role of repair inhibition. They found that satraplatin can potentiate the effects of radiation in human lung cancer cells, and that the mechanism of this effect is probably inhibition of DNA repair by satraplatin. Differences to other platinum drugs like cisplatin and carboplatin are indicated.

Vaisman et al. (Biochemistry 1999, 38, 11026) reported on the effects of DNA polymerases and high mobility group protein 1 on the carrier ligand specificity for translesion synthesis past platinum-DNA adducts, with respect to different platinum compounds.

Screnci et al. (Br J Cancer (2000) 82, 966) investigated the relationship between hydrophobicity, reactivity, accumulation and peripheral nerve toxicity of a series of platinum compounds. According to Screnci et al. the hydrophilicity of platinum drugs correlates with platinum sequestration in the peripheral nervous system, but not with neurotoxicity.

Wei et al. (J. Biol. Chem. 2001, 276, 38774) reported on the effect of ligands on the specific recognition of intrastrand platinum-DNA cross-links by high mobility group box and TATA-binding proteins, with respect to different platinum compounds.

Fokkema et al. (Biochem. Pharmacol. 2002, 63, 1989) analysed in detail the satraplatin-, JM118-, and cisplatin-induced cytotoxicities in relation to various parameters like platinum-DNA adduct formation, glutathione levels and p53 status in human tumor cell lines with different sensitivities to cisplatin. It was confirmed that satraplatin and JM118 can partially circumvent intrinsic and acquired resistance to cisplatin. At equimolar basis, satraplatin induced lower levels of platinum-DNA adducts in the cell lines tested compared to cisplatin.

Taken together, fundamental differences exist between satraplatin and other platinum agents, such as cisplatin. These differences are the basis, lead to or play a role in many of the different characteristics of satraplatin, including different pharmacokinetic properties, different efficacy, a different toxicology profile, different ADME properties and different mechanisms that lead to drug resistance, only to name a few.

Relevant In-Vitro & Pre-Clinical Investigations with Satraplatin

A number of preclinical investigations have been conducted using satraplatin during its development as a chemotherapeutic. In particular, the results of the following investigations have been published:

### Satraplatin in Models of Prostate Cancer and PSA Response

Wosikowski et al (AACR meeting: Basic, translational, and clinical advances in prostate cancer in Florida. Nov. 17-21, 2004) reported that treatment of prostate cancer cells with satraplatin or an active metabolite, JM118, resulted in tumor cell kill. The androgen-insensitive prostate cancer cell lines PC-3 and DU 145 was shown to be more sensitive to satraplatin than the androgen-sensitive LNCaP cell line. JM118 and JM518 were the most active metabolites of satraplatin and up to 16-fold more active than satraplatin.

Jung et al. (57. Jahrestagung der Deutschen Gesellschaft für Urologie, September 2005) and Wosikowski et al. (AACR meeting: Basic, translational, and clinical advances in prostate cancer in Florida. Nov. 17-21, 2004) reported that treatment of LNCaP cells for 42 hours with 12 μM satraplatin or 0.7 μM JM-118 resulted in a decrease in cell number (56% and 61% of control, respectively) and decrease in secreted PSA protein level (58% and 61% of control, respectively). However, there was no effect on PSA mRNA transcription (90% of control).

The pre-clinical evaluation of satraplatin and JM118 in human prostate cancer cell lines was also described by Wosikowski et al. on the Prostate Cancer ASCO meeting; San Francisco, Feb. 24-26, 2006.

Lamphere et al have reported: (i) the synergistic antitumor activity of the combination of satraplatin (S) and docetaxel (D) in H460 human non-small cell lung carcinoma (NSCLC) xenografted in nude mice (MCR Apr. 1-5, 2006 Washington, D.C. USA); and (ii) the antitumor activity of satraplatin in combination with paclitaxel in the H460 human non small cell lung carcinoma (NSCLC) xenografted in nude mice (AACR-NCI-EORTC International Conference on Molecular Targets and Cancer Therapeutics: Discovery, Biology, and Clinical Applications, Nov. 14-18, 2005, Philadelphia, Pa.).

Further, Lamphere et al have reported the synergistic antitumor activity of the combination of satraplatin (S) and paclitaxel (P) and the combination of satraplatin (S) and docetaxel (D) in prostate carcinoma models (ASCO 2006 Annual meeting, Atlanta, 24 Jun. 2006; AACR-NCI-EORTC International Conference on Molecular Targets and Cancer Therapeutics: Discovery, Biology, and Clinical Applications, Nov. 14-18, 2005, Philadelphia, Pa.).

PCT/EP2006/060615 describes that in various in-vitro and xenograft models of cancers including prostate cancer, satraplatin acts synergistically with certain other non-platinum-containing chemotherapeutic agents including taxanes such as paclitaxel (Taxol®) and docetaxel (Taxotere®).

Obermayr et al. (AACR-NCI-EORTC International Conference on Molecular Targets and Cancer Therapeutics: Discovery, Biology, and Clinical Applications, Nov. 14-18, 2005, Philadelphia, Pa.) reported the synergistic in vitro anticancer activity with sequential schedules of JM-118, a metabolite of satraplatin, in combination with erlotinib, paclitaxel or 5-FU.

EP 05024701.4 describes that in various in-vitro models of cancers, satraplatin acts synergistically with certain other non-platinum-based chemotherapeutic agents that include (i) inhibitors of receptors of the EGFR family, such as herceptin and erlotinib, and (ii) active pyrimidine analogues, such as gemcitabine, 5FU or prodrugs thereof.

### Satraplatin in Models of Resistant or Refractory Cancers

Cisplatin, carboplatin and oxaliplatin have shown clinical activity in testicular, ovarian, head and neck, small and non-small cell lung, and colon carcinomas. However, the effectiveness of these compounds has been limited due to intrinsic or acquired resistance. Proposed mechanisms of cisplatin resistance include increased DNA tolerance, reduced cellular accumulation of cisplatin and enhanced cellular detoxification of platinum complexes.

Various in-vitro studies show that satraplatin and JM-118 are able to overcome several of the mechanisms of resistance to cisplatin including those attributed to alterations in the DNA repair processes and platinum transport, and to mechanism associated with the resistance of cancers to certain non-platinum chemotherapeutic compounds. In particular: (i) satraplatin does not exhibit cross-resistance to a number of cisplatin-resistant cell lines; and (ii) resistance mechanisms that confer resistance to non-platinum based chemotherapeutic agents, i.e., taxanes, doxorubicin, vincristine, etoposide, mitoxantrone and camptothecin, generally do not confer cross-resistance to satraplatin or JM-118.

Kishimoto et al (2006 MCR meeting in Washington, 1-5 Apr. 2006) reported the differences in the mechanisms of resistance to cisplatin and to JM-118, an active metabolite of satraplatin.

Wosikowski et al. (MCR Annual Meeting in Anaheim, 16-20 Apr. 2005) reported the efficacy of satraplatin (JM216) and JM118 in certain drug resistant cells, and in combination with docetaxel.

WO 05/077385 describes that satraplatin is effective in the treatment of models of cancers and tumors that are resistant or refractory to certain other chemotherapeutic agents, including: (i) those cancers and tumors wherein the resistance mechanism is mediated by multidrug resistance mechanisms such as ABC transporters; (ii) cancers and tumors wherein the resistance mechanism is mediated by tubulin; and (iii) cancers and tumors wherein the resistance mechanism is mediated through topoisomerase. In particular, satraplatin was shown to be effective in models of cancer refractory or resistant to certain taxanes, including paclitaxel and docetaxel.

### Clinical Studies of the Efficacy of Satraplatin Against Hormone Resistant Prostate Cancer

A number of clinical studies have been conducted with satraplatin, and the results of these are summarised in Sternberg et al (BJU International, 2005, p. 990-994). Many of such clinical studies have investigated the pharmacology, toxicology and other safety of satraplatin in human subjects. Others clinical trials have tested the efficacy of satraplatin against a number of different cancers. The results of some of such clinical trials that set out to test the efficacy of satraplatin against hormone resistant prostate cancer have been published and are described below.

### Study 1

A pilot multicenter open-label phase II study to evaluate the efficacy and safety of satraplatin as a single-agent for first-line treatment of patients with hormone refractory prostate cancer was conducted in the U.S. (Peereboom et al: Proc. Am. Soc. Clin. Oncol. 16: 339a, 1997 & Latif et al; Investigational New Drugs 23: 79, 2005). Satraplatin was administered daily for 5 days every 4 weeks at a starting dose of 120 mg/m² per day. An interim analysis (Peereboom et al: Proc. Am. Soc. Clin. Oncol. 17: 314a, 1998) concluded that satraplatin is an active and convenient drug against HPRC and has manageable toxicities, whilst Latif et al concluded that although satraplatin had moderate activity in HRPC, it is associated with significant treatment-related toxicities in this patient population. The trial however was open-labeled, and hence any effect observed, is confounded by placebo effect.

### Study 2

A multicenter, randomized phase III study was designed to evaluate the efficacy and safety of satraplatin for first-line treatment of patients with HRPC (Sternberg et al: Proc. Am. Soc. Clin. Oncol. 22:395, 2003; Sternberg et al: Oncology 2005; 68:2-9). Patients were randomized between satraplatin 100 mg/m2 for 5 days plus prednisone 10 mg orally BID or prednisone alone. After 50 randomized patients, the trial was closed to further accrual by the sponsoring company. Median overall survival was 14.9 months (95% Cl: 13.7-28.4) on the satraplatin plus prednisone arm and 11.9 months (95% Cl: 8.4-23.1) on prednisone alone (hazard ratio, HR=0.84, 95% Cl: 0.46-1.55). A >50% decrease in prostate specific antigen (PSA) was seen in 9/27 (33.3%) in the satraplatin plus prednisone arm vs. 2/23 (8.7%) on the prednisone alone arm. Progression-free survival was 5.2 months (95% Cl: 2.8-13.7) on the satraplatin plus prednisone arm as compared to 2.5 months (95% Cl: 2.1-4.7) on the prednisone alone arm (HR=0.50, 95% Cl: 0.28-0.92). This randomized comparison of a combination of satraplatin and prednisone versus prednisone alone was suggestive of the antitumor activity of the combination. It was concluded that a role for satraplatin in the treatment of HPRC remains to be elucidated in an appropriate phase III setting. Other factors and parameters like pain progression and PSA levels were not followed up in this trial and no conclusion were made in these respects.

### Combination Studies of Satraplatin Against Cancer

As described above, satraplatin has shown increased efficiency when used in combination with certain other chemotherapeutics in a number of pre-clinical models of cancer. In certain early-phase clinical studies, satraplatin has been studied and when used in combination with the other therapeutics, including paclitaxel (Jones et al; Invest New Drugs 20: 55.61, 2002). In certain studies of the efficacy of satraplatin against hormone refractory prostate cancer (including that described as “Study 2 above), it has been used in combination with prednisone, a corticosteroid. Such trials have been conducted as so called “two-arm” trails, with one set of patients being treated with satraplatin plus prednisone, and the other set of patients treated with placebo plus prednisone. It has been considered unethical for clinical studies of HRPC to be conducted as “three-arm” trials; that is a trial in which a third arm is used to investigate and compare the efficacy of satraplatin alone (plus a placebo for the prednisone). Hence, it has been ethically impossible, and shall remain so, to investigate in clinical studies the potential synergy of such combinations by the use of such appropriate experimental design.

In summary, there is substantive preclinical in vitro and in vivo information for satraplatin, including information from in vitro or in vivo models for prostate cancer, and there are substantive results obtained in various clinical studies using satraplatin in various oncology indications, including as a first-line treatment of hormone refractory prostate cancer in combination with prednisone. Based on these suggestive studies, the SPARC (“Satraplatin and Prednisone Against Refractory Cancer”) phase III clinical study described in the Exemplification was started. However, while one could hope that the SPARC trial would be successful, one would not have had an expectation of actually achieving a statistically significant positive outcome in light of (i) the limitations in making predictions for clinical study results based on preclinical information, (ii) the known failure rate for phase III clinical trials, (iii) the complexity and severity of the underlying condition to be treated, and (iv) the limited success in treating hormone refractory prostate cancer (see below).

### Chemotherapy for Hormone Refractory Prostate Cancer

Until recently, the response of HRPC to cytotoxic agents, both singly and in combination, has been less than satisfactory (Pienta, 1994; Dawyson, 1993; Eisenberger, 1985; Yagoda, 1993). Objective disease regression occurs in approximately 10% to 20% of cases. Most responses are only partial. In a literature review of 3184 patients, the overall response rate (CR+PR) was only 7% (Yagoda, 1993). When the stable category was added, this figure increased by only 15% to 22%.

In the United States, there are two regimens approved for the first line treatment of HRPC: mitoxantrone plus a corticosteroid (e.g. prednisone) and docetaxel plus prednisone. Mitoxantrone is an anthracenedione that is effective, when combined with prednisone, in producing a palliative response using pain response criteria, in symptomatic patients (29% versus 12% with prednisone alone, p=0.01) (Tannock, 1996). The mitoxantrone plus corticosteroid regimen was thus approved as palliative treatment based on improvement in pain (Tannock, 1996). Despite the improvement in pain symptoms, however, no improvement in survival was observed with the combination therapy.

Prednisone therapy alone has been associated with an improved survival duration when compared to liarozole, a retinoic acid metabolism-blocking agent, for patients with hormone refractory prostate cancer (Oncology Drug Advisory Committee to the Food & Drug Administration, June 1997). Prednisone is normally used in combination with mitoxantrone at the dose of 5 mg twice daily for patients with symptomatic hormone refractory prostate cancer.

It has been recognized for many years that corticosteroids, such as prednisone, have a definite palliative and sometimes objectively beneficial effect on the clinical course of patients with hormone-refractory prostate cancer. Among the corticosteroids other than prednisone that have been investigated for use in therapies against HRPC are dexamethasone (Nelius et al., BJU Int. 2006, 98, 580-5; Odrazka et al., Oncol. Rep. 2005, 14, 1077-81; Storlie et al., Cancer 1995, 76, 96-100), hydrocortisone (Abratt et al., Ann. Oncol. 2004, 15, 1613-21; Kruit et al., Anticancer Drugs 2004, 15, 843-7), cortisone acetate (Ponder et al., Br. J. Cancer 1984, 50, 757-63), and prednisolone (Heidenreich, J. Urol. Urogynakol. 2004, 11, special edition 6 (edition for Austria), 15-19).

Estramustine is a mixed hormonal and alkylating agent. It is available in Europe, Australia and the U.S. for palliative treatment of patients with metastatic and/or progressive carcinoma of the prostate. Recent reports from clinical trials suggest that the combination of estramustine with either paclitaxel or docetaxel is well tolerated and produces a decrease of >50% in serum PSA levels in more than 50% of hormone refractory prostate cancer treated patients (Hudes, 1997; Petrylak, 1999; Hussain, 1999).

The taxanes, Taxol® (paclitaxel) and Taxotere® (docetaxel), have activity in hormone refractory prostate cancer when used alone or in combination with other cytotoxic agents (Hudes, 1997; Petrylak, 1999; Petrylak, 2004; Eisenberger, 2004). The results of two recent studies, SWOG 99-16 and TAX327, demonstrate a survival advantage in the docetaxel arms compared to mitoxantrone and prednisone (Petrylak, 2004; Eisenberger, 2004). Taxotere was recently approved by the FDA for use as first-line chemotherapy in patients with HRPC in combination with prednisone. The efficacy data generated through these two phase 3 randomized trials demonstrated for the first time a clinical benefit (survival advantage) for patients treated with chemotherapy for HRPC versus prednisone alone.

SWOG 99-16 was a randomized phase 3 trial of docetaxel and estramustine versus mitoxantrone and prednisone in men with androgen-independent prostate cancer (Petrylak, 2004). The median survival of men treated on the docetaxel/estramustine arm was 18 months and on the mitoxantrone/prednisone arm was 15 months. This difference was statistically significant (log rank p=0.008). The docetaxel/estramustine arm also demonstrated a superior median time to progression (6 months) compared to the mitoxantrone/prednisone arm (3 months), which was also statistically significant (log rank p<0.0001).

TAX327 was an international, multicenter phase 3 trial comparing docetaxel and prednisone, given either on an every 3 week schedule or a weekly schedule (5 of 6 weeks), to mitoxantrone and prednisone in patients with HRPC (Eisenberger, 2004; Dagher, 2004). The median survival in the every 3 weeks docetaxel arm was 18.9 months versus 16.5 months in the mitoxantrone arm. This difference was statistically significant (p=0.009). The median survival in weekly docetaxel schedule was 17.4 months. This was not statistically different compared to the mitoxantrone arm. However, when comparing the median survival of both docetaxel arms together (18.3 months) to the mitoxantrone arm, this difference was statistically significant (p=0.04).

In the TAX327 trial, the use of prednisolone, the active metabolite of prednisone, was allowed as a replacement of prednisone in case that oral tablets of prednisone were not marketed in the country where the trial took place (see Approval Package for Application Number 20-449/S-028: Medical Review(s) dated May 18, 2004). Correspondingly in Europe, the Committee for Medicinal Products for Human Use (CHMP) adopted a positive opinion to recommend the variation to the terms of the marketing authorisation for Taxotere (docetaxel) to add that Taxotere in combination with prednisone or prednisolone is indicated for the treatment of patients with hormone refractory metastatic prostate cancer.

Thus, chemotherapy is now an established treatment for HRPC, but the duration and response to first-line chemotherapy is limited, and a substantial number of patients will fail first-line therapy after an initial improvement of symptoms and modestly improved survival. There is a medical need for chemotherapeutic agents that may provide continued palliation and improved survival. Randomized trials must continue in order to identify new agents for the treatment of HRPC.

### The Challenges for any Future Hrpc Treatment

Due to the favorable results of the trials that led to approval of docetaxel (Taxotere®) for treatment of HRPC as first-line chemotherapy, it is anticipated that the number of patients treated with chemotherapy in the first-line setting of this disease will increase substantially. Once HRPC fails such first-line chemotherapy, subsequent treatment is needed for these patients. Currently there is no therapy approved for those patients for who the HRPC disease progresses despite such first-line chemotherapy; patients who have a median survival of only around 18 months. Considering the extent of the unmet medical need in HRPC, and following the approval of docetaxel for first-line chemotherapy the increasing number of HRPC patients in need of such second-line chemotherapy for HRPC, there is an urgent need for new therapies that can show significantly significant effects in clinical trials of hormone refractory prostate cancer in patients who were treated with a first-line cytotoxic chemotherapy regime.

Any therapy for second-line HRPC that shows statistically significantly efficacy in clinical trials, can lead to approval by FDA or other international drug-regulatory agencies, and the introduction and use of such therapy on a larger scale. Such use has the potential to materially improve the prospects of life-expectancy or life-quality of many men throughout the world. Any therapy that would enable such sick men to conduct the remainder of their life with as much dignity and convenience as possible, especially a therapy that could be practiced largely at home or in a more convenient setting, would have great significance and be of immense advantage to such men.

### SUMMARY OF THE INVENTION

We have invented that satraplatin in combination with prednisone is effective in the treatment of an individual suffering from metastatic hormone refractory prostate cancer, where such individual was treated with previous chemotherapy against such disease.

Thus, one aspect of the present invention relates to a method of treating an individual suffering from metastatic hormone refractory prostate cancer comprising administration of a therapeutically effective amount of satraplatin to said individual, wherein:
(a) said individual was treated with previous chemotherapy for metastatic hormone refractory prostate cancer; and
(b) said method further comprises the administration of prednisone to said individual in combination with said administration of satraplatin.

In another aspect, the invention relates to a packaged-pharmaceutical-product comprising a pharmaceutical composition that includes satraplatin, wherein said packaged-pharmaceutical-product further comprises instructions to conduct administration of a therapeutically effective amount of said satraplatin included in said pharmaceutical composition to an individual suffering from metastatic hormone refractory prostate cancer, wherein said instructions further include:
(a) an instruction to conduct said administration of satraplatin to an individual who was treated with previous chemotherapy for metastatic hormone refractory prostate cancer; and
(b) an instruction to conduct said administration of satraplatin in combination with administration of prednisone.

Such aspects include certain embodiments, wherein the packaged-pharmaceutical-product further comprises a second pharmaceutical composition that includes prednisone.

Another aspect of the present invention relates to a use of satraplatin for the preparation of a pharmaceutical composition including satraplatin for administration of a therapeutically effective amount of satraplatin to an individual suffering from metastatic hormone refractory prostate cancer, wherein:
(a) said individual was treated with previous chemotherapy for metastatic hormone refractory prostate cancer; and
(b) said individual is administered prednisone in combination with said administration of satraplatin.

Other features and advantages of the invention will be apparent from the following detailed description and from the claims.

### BRIEF DESCRIPTION OF THE DRAWINGS

FIG. 1.

Exemplary metabolites of satraplatin (JM216), depicting JM118, JM383, JM518, JM559 and JM149 (taken from Raynaud et al. 1996 Cancer Chemother. Phamacol. 38: 155-162).

FIG. 2.

Study schema of the “SPARC” trial (Satraplatin and Prednisone Against Refractory Cancer)

FIG. 3.

Representation of data to demonstrate significant efficacy of satraplatin, in combination with prednisone, against metastatic hormone resistant prostate cancer in patients having previous chemotherapy treatment. Kaplan Meier plot of Progression-Free Survival (as adjudicated by the IRC) for the ITT Population—SPARC Study: satraplatin (plus prednisone) arm, compared to placebo (plus prednisone) arm.

FIG. 4.

Demographic and Disease Characteristics —SPARC Study.

FIG. 5.

Kaplan Meier plot of Progression-Free Survival (as adjudicated by the IRC) for the subset of ITT Population who had received prior docetaxel —SPARC Study: satraplatin (plus prednisone) arm, compared to placebo (plus prednisone) arm.

FIG. 6.

Hazard ratios for PFS (and 95% confidence intervals) in various prognostic subsets—SPARC Study: satraplatin (plus prednisone) arm, compared to placebo (plus prednisone) arm. In the plot shown, estimated hazard ratio is depicted by a circle and the 95% confidence interval for the hazard ratio by the horizontal line.

FIG. 7.

(a) Kaplan Meier plot of Progression-Free Survival (as adjudicated by the IRC) for the subset of ITT Population who had disease-related pain at baseline (PPI score 1-5)—SPARC Study: satraplatin (plus prednisone) arm, compared to placebo (plus prednisone) arm; (b) Kaplan Meier plot of Progression-Free Survival (as adjudicated by the IRC) for the subset of ITT Population who were asymptomatic at baseline (PPI score 0)—SPARC Study: satraplatin (plus prednisone) arm, compared to placebo (plus prednisone) arm.

FIG. 8.

(a) Grade 3/4 haematological toxicity; (b) Grade 3/4 non-haematological toxicity.

FIG. 9.

Number of treatment cycles for patients in the SPARC Trial.

FIG. 10.

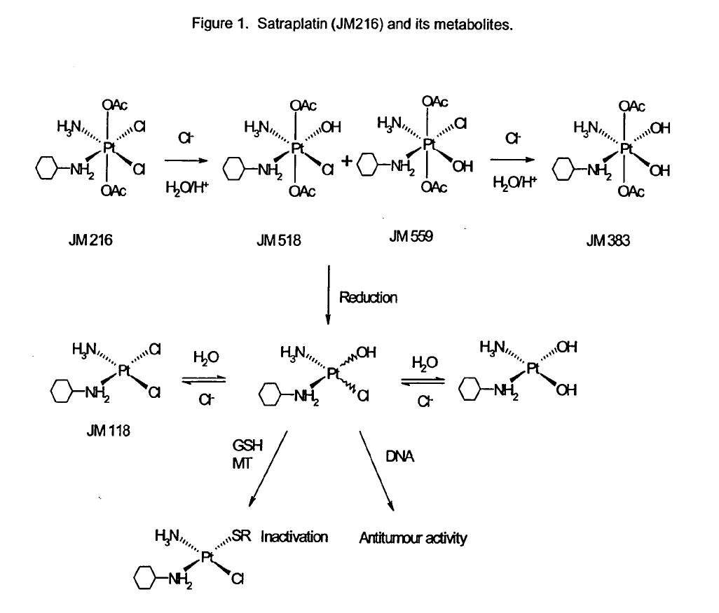

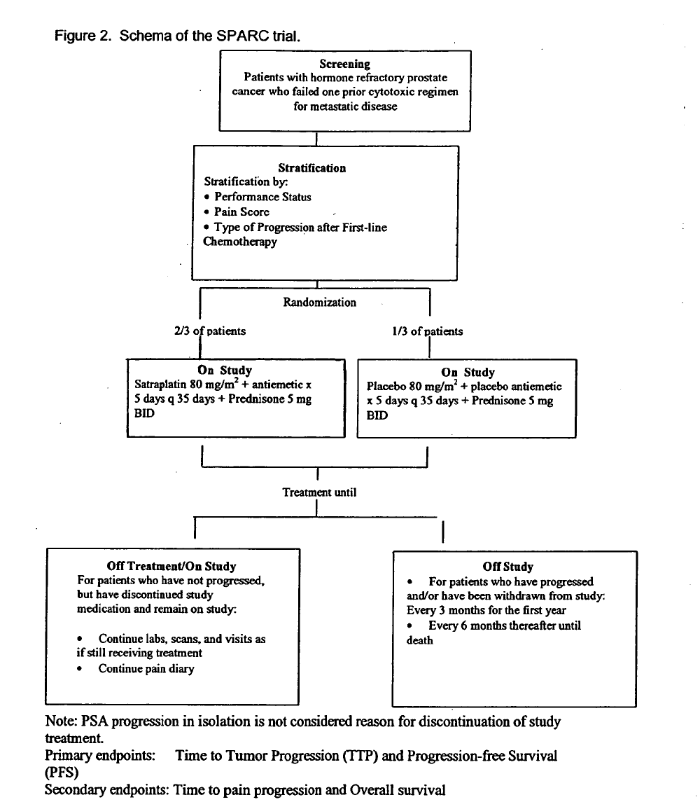

Time to Pain Progression Analysis for the Intent-to-Treat Population.

Exhibit A.

Public disclosure of results from SPARC trial.

Exhibit B.

Public disclosure II of results from SPARC trial.

### DETAILED DESCRIPTION OF THE INVENTION

### Definitions

The terms “administered”, “administration”, or “administering” a compound is understood by skilled artisans, such as clinical oncologists, and refers to providing a compound, such as a therapeutic agent including but not limited to satraplatin, prednisone or granisetron, to an individual in need of treatment by bringing such individual in contact with, or otherwise exposing such individual to, such compound. Compounds may be administered as a pharmaceutical composition or formulation.

The term “antiemetic agent” is understood by skilled artisans, such as clinical oncologists, and refers to any anti-emetic agent known to the skill artisan, including, but not limited to, serotonin-3 receptor antagonists like granisetron, dolasetron, ondansetron and tropisetron, NK1 receptor antagonists, antihistamines such as cinnarizine, cyclizine and promethazine, histamine H2 receptor antagonists such as ranitidine (Zantac), phenothiazines such as chlorpromazine, droperidol, haloperidol, methotrimeprazine, perphenazine, trifluoperazine and prochlorperazine, domperidone, and metoclopramide.

The term “chemotherapy” is understood by skilled artisans, such as clinical oncologists, and refers to the treatment of cancer with chemical compounds that have a specific toxic effect upon the cancer, e.g. by interfering with cell reproduction. By way of non-limiting example, compounds useful for chemotherapy of metastatic prostate cancer include taxanes such as paclitaxel and docetaxel, mitoxantrone, viniblastine and estramustine.

The term “in combination” when used in reference to administration is understood by skilled artisans, such as clinical oncologists, and refers to the essentially simultaneous or sequential administration of at least two compounds, including but not limited to the two compounds satraplatin and prednisone. Such compounds may be administered sequentially with each other, with the term “in combination” not being limited in the sequence of administration; encompassing when a compound is administered either prior to or after administration of another compound. By way of non-limiting example, satraplatin and prednisone are considered to be administered “in combination” during the treatment regime using such compounds that is set out with in the exemplification. A compound may also be administered “in combination” with another compound when both are administered essentially at the same time or simultaneously, including when appropriate when both compounds are formulated as single dosage form.

The term “corticosteroid” is understood by skilled artisans, such as clinical oncologists, and refers to a family of semisynthetic and synthetic compounds that mimic the anti-inflammatory effects of cortisol. The most commonly prescribed agents include cortisone acetate, hydrocortisone, prednisone, dexamethasone, and prednisolone.

The term “cytotoxic” is understood by skilled artisans, such as clinical oncologists, and refers to the property of e.g. a compound to be toxic to cells, including the ability to kill a cell.

The term “cytotoxic chemotherapy regime” is understood by skilled artisans, such as clinical oncologists, and refers to a treatment procedure or regime that uses, performs or requires chemotherapy that involves at least one compound that is believed to be cytotoxic, e.g. by administering a certain dosage or dosages of such compound at, or over, a defined period of time, in one or more cycles, with or without concomitant or sequential administration of additional cytotoxic compounds, or, for example, analgesic or antiemetic compounds.

The term “bone pain” is understood by skilled artisans, such as clinical oncologists, and also by patients, and refers herein to a pain commonly associated with metastatic cancer such as metastatic prostate cancer, and is felt in (or has the sensation of stemming from) bones of the patient. Bone pain can be referred to as “ostealgia” or “osteodynia” by skilled artisans. Without being bound by theory, the pain occurs due to the disruption of the balance of normal cellular activity in the bones, causing damage to the bone tissue. Normal bone is constantly being remodeled, or broken down and rebuilt. Cancer cells that have spread to the bone disrupt this balance between the activity of osteoclasts and osteoblasts, resulting in either weakened or excessively built-up bone. This damage can either stretch the periosteum or stimulate nerves within the bone, and is a major cause of such pain.

The term “lymph pain” is understood by skilled artisans, such as clinical oncologists, and also by patients, and refers herein to a pain or discomfort felt in (or has the sensation of stemming from) a lymph node. For pain associated with metastatic cancer, such pain can felt in lymph nodes that are regional or distant to the primary cancer or tumour. In the case of prostate cancer for example, regional lymph nodes can be those nodes found in the groin, while distant lymph nodes can be those in the neck or under-arm region. Lymph nodes are often one of the first organs of the body in which metastases of a primary cancer are found. Without being bound by theory, metastases that establish in or near lymph nodes can cause swelling of or pressure on such nodes. Such swelling or pressure can cause discomfort and pain.

As used herein the term “packaged-pharmaceutical-product” refers to any packaging system for storing and dispensing individual doses of medication, including such a system storing for and dispensing to the patient who ultimate consumes the medication. The packaged-pharmaceutical-product can contain sufficient daily dosage units appropriate to the treatment period or regime, or in amounts which facilitate the patient's compliance with the regimen. In certain embodiments, the packaged-pharmaceutical-product comprises one or more vessels that include the compound to be used in the treatment according to the present invention. Such vessel can be a unit dosage form such as a capsule or pill, or may be a container such as a bottle, vial or syringe. The compound may be provided in the vessel in a pharmaceutically acceptable form or may be provided, for example, as a lyophilized powder. In further embodiments, the packaged-pharmaceutical-product may further include a solvent to prepare the compound for administration. In certain embodiments, the compound may be already provided in a delivery device, such as a syringe, or a suitable delivery device may be included in the pack. The packaged-pharmaceutical-product may comprise pills, liquids, gels, tablets, dragees or the pharmaceutical preparation in any other suitable form. The packaged-pharmaceutical-product may contain any number of daily pharmaceutical dosage units, or a number of dosage units sufficient for multiple days of a treatment regime. The package may be of any shape, and the unit dosage forms may be arranged in any pattern, such as circular, triangular, trapezoid, hexagonal or other patterns. One or more of the doses or subunits may be indicated, for example to aid the doctor, pharmacist or patient, by identifying such dose or subunits, such as by employing color-coding, labels, printing, embossing, scorings or patterns. The packaged-pharmaceutical-product may also comprise instructions for the patient, the doctor, the pharmacist or any other related person.

Some embodiments comprise the administration of more than one active ingredient, including compounds as disclosed herein. Such administration may occur concurrently or sequentially. The active ingredients may be formulated together such that one administration delivers both components. Alternatively the active ingredients may be formulated separately. In certain such embodiments, the packaged-pharmaceutical-product may comprise: (i) a compound used in the present invention and any the other pharmaceutical ingredient in a single formulation (i.e., they are formulated together), or (ii) such compound used in the present invention and the other pharmaceutical ingredient in individual formulations (i.e., they are formulated separately). Each formulation may comprise a compound used in the present invention and any other pharmaceutical ingredient in individual dosage amounts (in approximately equal or unequal amounts).

As used herein, the term “instructions” means a product label and/or documents describing relevant materials, methodologies or information pertaining to assembly, preparation or use of a packaged-pharmaceutical-product or any component contained therein. For example, such instructions may include details of the indications and usage of such component, therapeutic procedure or regime to be followed, with appropriate doses and mode of administrations that provide therapeutically effective amounts of any compounds used in such therapeutic regime, dosage modifications, warnings and precautions and other information pertinent for the safe and effective application of the packaged-pharmaceutical-product in the area of health-care. These materials, methodologies or information may include any combination of the following: background information, steps or procedures to follow, list of components, proposed dosages for therapeutically effective amounts, warnings regarding possible side effects, instructions for administering the drug, technical support, and any other related documents. Instructions can be supplied in printed form, such as a package label or a package insert. Non-limiting example of “instructions” in the form of a package insert, can be obtained from the Center for Drug Evaluation and Research of the U.S. FDA, including via http://www.accessdata.fda.gov/scriptslcder/drugsatfda/index.cfm. Such form of instructions can be required to be approved before use by a drug regulatory authority, such as the FDA, and only after appropriate clinical trials have been conducted that show significantly significant effects following treatment with the drug. Alternatively, instruction may also be stored in electronic form, e.g., on a computer-readable storage medium such as a computer-readable memory device, a centralized database, magnetic media such as hard disks, floppy disks, and magnetic tape; optical media such as compact discs, CD-ROMs and holographic devices; magneto-optical media such as floptical disks; and hardware devices that are specially configured to store and execute program code, such as application-specific integrated circuits (ASICs), programmable logic devices (PLDs) and ROM (read only memory) and RAM (random access memory) devices. Instructions may comprise a web address of an internet website from which more detailed instructions may be downloaded, or a recorded presentation. Instructions can contain one or multiple documents or future updates.

The term “taxane” is understood by skilled artisans, such as clinical oncologists, and is meant to include any member of the family of terpenes, including, but not limited to paclitaxel (Taxol) and docetaxel (Taxotere), which were derived primarily from the Pacific yew tree, Taxus brevifolia, and which have activity against certain tumors, particularly breast, lung and ovarian tumors (See, for example, Pazdur et al. Cancer Treat Res. 1993. 19:3 5 1; Bissery et al. Cancer Res. 1991 51:4845). In particular embodiments of the methods, uses and packaged-pharmaceutical-products of the present invention, taxanes are paclitaxel, docetaxel, deoxygenated paclitaxel, TL-139 and their active derivatives. See Annu. Rev. Med. 48:353-374 (1997).

The term “taxane” as used herein includes naturally occurring or partly or fully chemically synthesized paclitaxel, which is sold as TAXOL® by Bristol-Myers Oncology, as well as terpene compounds derived from, or related to, paclitaxel, or other derivatives thereof, including deoxygenated paclitaxel compounds, such as those described in U.S. Pat. Nos. 5,440,056 and 4,942,184, which are herein incorporated by reference. Paclitaxel has been approved for clinical use in the treatment of refractory ovarian cancer in the United States (Markman et al., Yale Journal of Biology and Medicine, 64:583, 1991; McGuire et al., Ann. Intern. Med., 111:273, 1989). It is effective for chemotherapy for several types of neoplasms including breast (Holmes et al., J. Nat. Cancer Inst., 83:1797, 1991) and has been approved for treatment of breast cancer as well. It is a potential candidate for treatment of neoplasms in the skin (Einzig et al., Proc. Am. Soc. Clin. Oncol., 20:46, 2001) and head and neck carcinomas (Forastire et al. Sem. Oncol., 20:56, 1990). The compound also shows potential for the treatment of polycystic kidney disease (Woo et al, Nature, 368:750, 1994), lung cancer and malaria. Docetaxel (N-debenzoyl-N-tert-butoxycarbonyl-10-deacetyl paclitaxel) is produced under the trademark TAXOTERE® by Sanofi-Aventis. In addition, other taxanes are described in “Synthesis and Anticancer Activity of Taxol other Derivatives,” D. G. 1. Kingston et al., Studies in Organic Chemistry, vol. 26, entitled “New Trends in Natural Products Chemistry” (1986), Atta-ur-Rahman, P. W. le Quesne, Eds. (Elvesier, Amsterdam 1986), pp 219-235 are incorporated herein. Various taxanes are also described in U.S. Pat. No. 6,380,405, the entirety of which is incorporated herein.

Without being bound by theory, taxanes exert their cytotoxic effect on cells, including cancer and tumour cells, by binding to tubulin, thereby causing the formation of unusually stable microtubules. The ensuing mitotic arrest triggers the mitotic spindle checkpoint and results in apoptosis. Other mechanisms that mediate apoptosis through pathways independent of microtubule dysfunction have been described as well, including molecular events triggered by the activation of Cell Division Control-2 (cdc-2) Kinase, phosphorylation of BCL-2 and the induction of interleukin 1β (IL-1β) and tumour necrosis factor-α (TNF-α). Furthermore, taxanes have been shown to also exert anti-tumour activity via mechanisms other than the direct activation of the apoptotic cascade. These mechanisms include decreased production of metalloproteinases and the inhibition of endothelial cell proliferation and motility, with consequent inhibition of angiogenesis.

The term “therapeutically effective amount” of a compound, including an active ingredient, therapeutic agent or drug, is understood by skilled artisans, such as clinical oncologists, and refers to an amount of a compound to be administered to an individual in need of therapy or treatment, as required by any particular dosage, therapeutic or administration regimen or procedure, and as according to clinically acceptable standards for the disease, disorder, symptom or condition to be treated, or at a reasonable benefit/risk ratio applicable to such treatment. In the case of metastatic hormone resistant prostate cancer, such amount is reasonably in accordance with the amount of such compound that has been demonstrated to have the desired therapeutic effect with statistic significance in a clinical trial, such as in the SPARC phase III clinical trial shown herein in the Exemplification.

It is well known to anyone of ordinary skill that for a given individual, the therapeutically effective amount, dosage form and timing and form of administration of such therapeutically effective amount, will be determined by a qualified physician, or other person having appropriate knowledge and qualification, based on one or more of: (i) the dosage, dosage form and timing and form of administration used in the clinical study that has demonstrated the statistically significant clinical efficacy for the respective treatment, (ii) recommendations for the dosage, dosage form and timing and form of administration provided in any instructions provided with the pharmaceutical form of the compound, including the approved product label or insert for such treatment, and (iii) factors specific for such individual that may influence the actual dose or amount to be administered to the individual. Thus, the dosage administered will, of course, vary depending upon known factors such as the pharmacodynamic characteristics of the particular compound and its mode and route of administration; age, sex, health, weight, body surface area, neutrophil count, of the individual to be treated; nature and extent of symptoms; kind of concurrent treatment, frequency of treatment and the effect desired. Furthermore, scientific or medical publications or reports on additional clinical studies, especially those related to efficiency or safety of the compound when used in other setting, may influence the determination of a dosage, dosage form, or and timing and form of administration in order to determine an amount reasonably expected to be a therapeutically effective amount for any given individual.

The term “time to disease progression”, is understood by skilled artisans, such as clinical oncologists, and refers to the time from initiation of a particular therapy or treatment regime or protocol for an individual, such as administration of satraplatin to patients suffering from metastatic hormone resistant prostate cancer, to when disease progression is then first observed in such individual, as determined from one or more symptoms or characteristics of the individual. Time to disease progression can be abbreviated to “TTP”. By way of example, “time to disease progression” in the SPARC trial was used to refer to the time period described in section 10.3.1 of the clinical protocol.

The term “progression-free survival” is also understood by skilled artisans, such as clinical oncologists, and refers to the time from initiation of a particular therapy or treatment regime or protocol for an individual, such as administration of satraplatin to patients suffering from metastatic hormone resistant prostate cancer, to the earlier of: (i) when disease progression is then first observed in such individual, as determined from one or more symptoms or characteristics of the individual; or (ii) death of the individual. Progression-free survival can be abbreviated to “PFS”. By way of example, “progression-free survival” in the SPARC trial was used to refer to the time period described in section 10.3.2 of the clinical protocol. The term “time to pain progression” is also understood by skilled artisans, such as clinical oncologists, and refers to the time from initiation of a particular therapy or treatment regime or protocol for an individual, such as administration of satraplatin to patients suffering from metastatic hormone resistant prostate cancer, to when pain-related progression is then first observed in such individual. Time to pain progression can be abbreviated to “TPP”. By way of example, “time to pain progression” in the SPARC trial was used to refer to the time period described in section 10.7.3 of the clinical protocol.

The term “overall survival” is also understood by skilled artisans, such as clinical oncologists, and refers to the time from initiation of a particular therapy or treatment regime or protocol for an individual, such as administration of satraplatin to patients suffering from metastatic hormone resistant prostate cancer, to death of such individual.

The term “chemotherapy holiday” is also understood by skilled artisans, such as clinical oncologists, and refers to the use of intermittent chemotherapy—whereby during the chemotherapy (such as chemotherapy with docetaxel) breaks or “holidays” in the chemotherapy are given (for example, Br J Cancer 2003; 89:968-970). Although chemotherapy drugs can be effective, side effects can accumulate when such drugs are used for prolonged periods of time, and it is unrealistic to continue the treatment indefinitely. Indeed, patients are often unable to tolerate continuous ongoing chemotherapy, such as therapy with docetaxel, and chemotherapy can be administered intermittently: patients take a break (a “chemotherapy holiday”) from treatment and resume at a specified point in the future.

### Particular Embodiments

In certain embodiments, prednisone is administered in a therapeutically effective amount.

In one embodiment, the individual to be treated in accordance with the present invention has a diagnosis of Stage D2 adenocarcinoma of the prostate that is unresponsive to hormone therapy.

In another embodiment, the individual has failed treatment with previous chemotherapy.

In yet another embodiment, the individual has taken a chemotherapy holiday from said previous chemotherapy.

In certain embodiments, the chemotherapy was a cytotoxic chemotherapy regime.

In certain embodiments, the individual has suffered disease progression or PSA progression after a minimum of two courses of one prior cytotoxic chemotherapy regime for metastatic hormone refractory prostate cancer.

In another embodiment, the chemotherapy or cytotoxic chemotherapy regime used a compound selected from mitoxantrone, viniblastine, estramustine and a taxane, including embodiments where the compound is a taxane, including paclitaxel and docetaxel.

In a certain embodiment, the taxane is docetaxel.

In a certain embodiment, the previous chemotherapy did not use a platinum-containing compound, including satraplatin. In a related embodiment, the individual has not had prior treatment with a platinum-containing compound, including satraplatin.

In an alternative embodiment, the previous chemotherapy did not use mitoxantrone in combination with a corticosteroid.

In yet another embodiment, the individual is administered satraplatin orally at a dose of between about 30 mg/m² and about 140 mg/m² per day over between 3 and 7 days, including administration at a dose of between about 40 mg/m² and about 100 mg/m², or at a dose of between about 50 mg/m² and about 90 mg/m², in each case per day, over between 3 and 7 days.

In another embodiment, the individual is administered satraplatin orally at a dose of about 40 mg/m² per day, at a dose of about 60 mg/m², or at a dose of about 80 mg/m², in each case over between 3 and 7 days.

In a certain embodiment, the actual amount or dose of satraplatin administered orally to the individual is rounded to the nearest 10 mg.

In a certain embodiment, the individual is administered satraplatin daily for about five consecutive days, with the cycle repeated about every 35 days. In an alternative certain embodiment, the individual is not administered satraplatin with such five consecutive days for no more than two days, and satraplatin is administered for a further number of days equal to the such number of days the individual is not administered satraplatin. In another certain embodiment, the cycle is repeated after about 38 days.

In another embodiment, the individual is examined after an appropriate period of time following the administration of satraplatin. Such examination can include the examination or assessment of one or more of: History and Physical (H&P), Weight and Performance Status (“PS”), Toxicity Assessment, PSA, Bone scan, Tumor Assessment, Complete Blood Count (CBC), platelets, absolute neutrophil count (“ANC”), Serum Chemistry, Chest X-ray, Electrocardiogram, Present Pain Intensity (“PPI”) Diary or Analgesic Diary. Such examinations or assessments can be conducted using methodologies that are known to skilled artisans, such as clinical oncologists, for example, as described in the Exemplification.

In a certain embodiment, the individual is examined or assessed for at least one of neutropenia, thrombocytopenia or non-hemotologic toxicity.

In one embodiment, the individual is retreated with satraplatin if the absolute neutrophil count is greater than or equal to about 1.5×10⁹/L, and platelets are more than or equal to about 100×10⁹/L. In another embodiment, the individual is retreated if no non-hematological toxicity that is ascribed to the therapy resolves to base line of greater than or equal to grade 1, for example as graded according to the NCI Common Toxicity Criteria Version 2.0. In certain such embodiments, the individual is retreated with a dose of satraplatin at about 100 mg/m² per day.

In an alternative embodiment, the individual is retreated with a decreased dose of satraplatin if the absolute neutrophil count is less than about 1.5×10⁹/L, platelets are less than about 100×10⁹/L, or the individual shows non-hematological toxicity that is ascribed to the therapy. In certain such embodiments, the individual is retreated with a reduced dose of satraplatin at about administered a dose of satraplatin at about 60 mg/m² or 40 mg/m² per day.

In a particular embodiment of the invention, the individual is not retreated with satraplatin if upon examination or assessment if one or more of the following observations are made in the individual: (i) neutropenia (neutrophil count is less than about 0.5×10⁹/L) or thrombocytopenia (platelets less than about 25×10⁹/L) despite dose reduction to 40 mg/m² per day; (ii) grade 3 or 4 hepatic (lasting >7 days), renal, cardiac, pulmonary, or neurological toxicity; or (iii) grade 4 vomiting or diarrhea that cannot be controlled by medical treatment and that occurs after one dose reduction.

In another embodiment, no food is taken by the individual for at least about one hour before, and for at least about 2 hours after administration of satraplatin.

In yet another embodiment, administration of satraplatin is to the individual on an empty stomach.

In other embodiment, the individual is administered prednisone orally with an amount of between 2 mg and 10 mg twice per day, including with an amount of 5 mg twice per day.

In certain embodiments, the individual is administered prednisone orally about one hour prior to administration of satraplatin orally and about eight hours after administration of satraplatin orally.

In certain embodiments, the individual is administered prednisone in the morning and the evening on those days of a cycle when satraplatin is not administered. In particular such embodiments, the individual is administered prednisone in the morning and the evening without administration of satraplatin for about 30 consecutive days.

In certain embodiment of all aspects of the invention, the individual is administered a number of cycles of treatment, wherein such number is greater than 3, 4 or 5 cycles. In particular such embodiments, such number is greater than 7, 9 or 11 cycles. In other particular embodiments, such number is greater than 16, 18 or 20 cycles. In yet other particular embodiments, such number is greater than 5, 9 or 16, but less than 90, 60 or 30 cycles, including where such number of cycles is between 5 and about 35 cycles, or between 17 and about 28 cycles. In particular such embodiments, the individual has one or more cycle delayed by one week or more, including 1, 2 or 3 such cycles delayed by about 1 week. In other particular such embodiments, the individual has two or more cycle delayed by one week or more, including by about 1 week., including 2, 3 or 4 cycles being so delayed.

In yet another embodiment, the individual is further administered an antiemetic agent on the same day of administration of satraplatin, including embodiments wherein the antiemetic agent is administered about one hour prior to administration of satraplatin orally and about eight hours after administration of satraplatin orally.

In certain embodiments, the antiemetic agent is administered in a therapeutically effective amount.

In a related embodiment, the individual is premedicated with an antiemetic agent.

In certain embodiments, the antiemetic agent is a 5-HT3 blocker or inhibitor, including ondansetron, tropisetron, or dolasetron, and further including embodiments wherein the antiemetic agent is granisetron. In certain of these embodiments, granisetron is administered orally with an amount of between 0.2 mg and 5 mg, including embodiments where granisetron is administered orally with an amount of 1 mg.

In another embodiment, the method of the present invention comprises the steps of: (a) to said individual, on each of days 1 to 5, the administration of prednisone (5 mg) and antiemetic agent (1 mg) orally, followed after about 1 hour by the administration of satraplatin orally at a dose of about 80 mg/m², followed after about 8 hours by the administration of prednisone (5 mg) and antiemetic agent (1 mg) orally; (b) to said individual, on each of days 6 to 35 the administration of prednisone (5 mg) twice daily in the morning and evening; and (c) repeating (a) and (b) at least one time.

In a certain embodiment, the instructions included in the packaged-pharmaceutical-product of the present invention comprise instructions to conduct the steps of: (a) to said individual, on each of days 1 to 5, the administration of prednisone (5 mg) and antiemetic agent (1 mg) orally, followed after about 1 hour by the administration of satraplatin orally at a dose of about 80 mg/m², followed after about 8 hours by the administration of prednisone (5 mg) and antiemetic agent (1 mg) orally; (b) to said individual, on each of days 6 to 35 the administration of prednisone (5 mg) twice daily in the morning and evening; and (c) repeating (a) and (b) at least one time.

An embodiment of the use of the present invention is further characterised as: (a) to said individual, on each of days 1 to 5, prednisone (5 mg) and antiemetic agent (1 mg) is administered orally, followed after about 1 hour by the administration of satraplatin orally at a dose of about 80 mg/m², followed after about 8 hours by the administration of prednisone (5 mg) and antiemetic agent (1 mg) orally; (b) to said individual, on each of days 6 to 35 prednisone (5 mg) is administered twice daily in the morning and evening; and (c) repeating (a) and (b) at least one time.

In certain such embodiments, the individual is examined or assessed for at least one of neutropenia, thrombocytopenia or non-hemotologic toxicity after (b) and before (c). In a particular such embodiment, (c) is conducted if the absolute neutrophil count is greater than or equal to about 1.5×10⁹/L, and platelets are more than or equal to about 100×10⁹/L.

In other such embodiments, the satraplatin is administered to the individual on an empty stomach. In a related such embodiment, the individual had not received food for one hour before or two hours after the administration of satraplatin.

In certain embodiments of all aspects of the invention, the administration of satraplatin results in an extension, elongation or prolongation of the time to disease progression.

In certain embodiments of all aspects of the invention, the administration of satraplatin results in an extension, elongation or prolongation of the progression-free survival.

In a particular embodiment, the administration of satraplatin results in a extension, elongation or prolongation of the progression-free survival of between about 5 weeks to about 50 weeks In another particular embodiment, such extension, elongation or prolongation of progression-free survival is between about 8 weeks and about 25 weeks, including a extension, elongation or prolongation of the progression-free survival of between about 10 weeks to about 20 weeks.

In a certain embodiment, the administration of satraplatin results in a progression-free survival of between about 10 weeks to about 50 weeks. In a particular such embodiment, such progression-free survival is between about 15 weeks and about 40 weeks, including a progression-free survival of between about 20 weeks to about 35 weeks.

In another certain embodiment, the administration of satraplatin results in between about 10% to about 80% lower risk of the progression-free survival. In a particular such embodiment, such lower risk of progression-free survival is between about 15% to about 50%, while in another particular such embodiment, such lower risk of progression-free survival is between about 20% to about 35%, including a lower risk of about 30% to about 35% of the progression-free survival.

In certain embodiments of all aspects of the invention, the administration of satraplatin results in an extension, elongation or prolongation of the overall survival.

In certain embodiments of all aspects of the invention, the individual suffering from metastatic hormone refractory prostate cancer is suffering from pain.

In certain such embodiments, the pain is cancer-related pain.

In other such embodiments, the pain is pain associated with metastatic hormone refractory prostate cancer.

In certain such embodiments, the pain is caused by metastases.

In certain such embodiments, the pain is bone pain or lymph pain.

In certain such embodiments, the administration of satraplatin results in relief or alleviation of the pain, in stable, or in stabilization of, pain, or in an extension, elongation or prolongation of the time to pain progression.

In a particular embodiment, the administration of satraplatin results in a extension, elongation or prolongation of the time to pain progression of between about 5 weeks to about 50 weeks In another particular embodiment, such extension, elongation or prolongation of time to pain progression is between about 10 weeks and about 30 weeks, including a extension, elongation or prolongation of the time to pain progression of between about 15 weeks to about 20 weeks.

In certain embodiment, the administration of satraplatin results in a time to pain progression of between about 20 weeks to about 100 weeks In a particular such embodiment, such time to pain progression is between about 30 weeks and about 80 weeks, including a time to pain progression of between about 40 weeks to about 60 weeks.

In another certain embodiment, the administration of satraplatin results in a lower risk of pain progression of between about 15% to about 50%. In a particular such embodiment, the administration of satraplatin results in a lower risk of pain progression of between about 20% to about 40%, including a lower risk of pain progression of between about 30% to about 35%.

In yet another certain embodiment, the administration of satraplatin results in relief of pain, including embodiments where such relief lasts for between about 15 weeks to about 80 weeks, between about 25 weeks to about 60 weeks or between about 30 weeks to about 56 weeks.

In other such embodiments, the individual does not show an increase in PPI score or analgesic consumption. In a particular such embodiment, the individual does not experience an increase cancer related pain, of at least one point from baseline or at least 2 points compared with the nadir, observed for at least 2 weeks (based on 2 or more consecutive weekly PPI determinations), or the individual does not show an increase in average analgesic score of greater than 25% compared with base line that is maintained for more than 2 consecutive weeks. In particular such embodiments of the invention, the individual shows a decrease in PPI score or analgesic consumption.

In another embodiment of all aspects of the invention, the individual does not show: (i) a decrease in ECOG performance status of greater than 2 units compared to baseline attributable to cancer for longer than about two weeks; and (ii) weight loss of greater than 10% of initial body weight attributable to cancer. In particular such embodiments, the individual shows an increase in ECOG performance status or a weight gain.

In certain embodiments of all aspects of the invention, the individual: (i) suffers from Stage D2 adenocarcinoma of the prostate that is unresponsive to hormone therapy; (ii) has shown progression of such disease after 1 prior cytotoxic chemotherapy regimen (prior prednisone therapy permitted); (ii) is classified as Eastern Cooperative Oncology Group (ECOG) performance status ≦2; (iii) has no history of major gastrointestinal surgery or conditions that may impair absorption; (iv) shows no symptoms of active gastric or duodenal ulcer; and/or (v) does not suffer from uncontrolled insulin-dependent diabetes.

In other certain embodiments of all aspects of the invention, the individual is an asymptomatic patient, including patients that are asymptomatic for pain (for example with a PPI score of 0).

In yet other certain embodiments of all aspects of the invention, the individual has not shown progression of HRPC as determined by pain progression, while in another alternative embodiment of all aspects of the invention, the individual has not shown progression of such disease as determined by PSA level, increase in PSA or rate of (“velocity”) of PSA increase. In alternative embodiments of all aspects of the invention, the individual has HRPC that has progressed as determined by pain progression, while in another alternative embodiment of all aspects of the invention, the individual has shown progression of such disease as determined by PSA level, increase in PSA or rate of (“velocity”) of PSA increase.

In yet other embodiments of all aspects of the invention, the individual is older than 50 years, is between about 50 and about 95 years or is between about 60 and about 90 years, including individuals older than 65 years and younger than about 85 years.

In yet another embodiment of all aspects of the invention, the individual is administered satraplatin together with another therapy, such as chemotherapy, including embodiments where the other therapy and the satraplatin is administered is within about 35 days, 28 days, 14 days, 7 days or 2 days of each other. In particular embodiments, the other therapy and the satraplatin is administered on the same day, or effectively at the same place. In certain embodiments, the other therapy uses active ingredients to relieve pain, including bisphosphonates or opioid analgesics, or to control or ameliorate diarrhea. In certain other embodiments, the other therapy is chemotherapy that does not use a compound that is a taxane, such as paclitaxel or docetaxel, mitoxantrone, viniblastine or estramustine. In other certain embodiments, the chemotherapy is radiation therapy or uses a radionuclide. In yet another embodiment, the chemotherapy uses a compound selected from: altretamine, busulfan, chlorambucil, cyclophosphamide, ifosfamide, mechlorethamine, melphalan, thiotepa, cladribine, fluorouracil, floxuridine, capecitabine, gemcitabine, thioguanine, pentostatin, methotrexate, 6-mercaptopurine, cytarabine, carmustine, lomustine, streptozotocin, carboplatin, cisplatin, oxaliplatin, picoplatin, LA-12, iproplatin, tetraplatin, lobaplatin, fludarabine, aminoglutethimide, flutamide, goserelin, leuprolide, megestrol acetate, cyproterone acetate, tamoxifen, anastrozole, bicalutamide, dexamethasone, diethylstilbestrol, prednisone, bleomycin, dactinomycin, daunorubicin, doxirubicin, erlotinib, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and in yet another embodiment, the chemotherapy uses a compound that is a non-small molecule therapeutic, including but not limited to antibodies, e.g., 1D09C3 and other anti-HLA-DR antibodies as described in WO 01/87337 and WO 01/97338, Rituxan as described in U.S. Pat. Nos. 5,736,137, 5,776,456, 5,843,437, 4D5, Mab225, C225, Daclizumab (Zenapax), Antegren, CDP 870, CMB401, MDX-33, MDX-220, MDX477, CEA-CIDE, AHM, Vitaxin, 3622W94, Therex, 5G1.1, IDEC-131, HU-901, Mylotarg, Zamyl (SMART M195), MDX-210, Humicade, LymphoCIDE, ABX-EGF, 17-1A, Trastuzumab (Herceptin®, rhuMAb), Epratuzumab, Cetuximab (Erbitux®), Pertuzumab (Omnitarg®, 2C4), R3, CDP860, Bevacizumab (Avastin®), tositumomab (Bexxar®), Ibritumomab tiuxetan (Zevalin®), M195, 1D10, Hu1D10 (Remitogen®, apolizumab), Danton/DN1924, an “HD” antibody such as HD4 or HD8, CAMPATH-1 and CAMPATH-1H or other variants, fragments, conjugates, derivatives and modifications thereof, or other equivalent compositions with improved or optimized properties, and proteins or peptides, e.g., those described in Trends in Biotechnology (2003), 21(12), p. 556-562.

In one embodiment, the other therapy is chemotherapy that uses a compound that is a taxane, such as paclitaxel or docetaxel, mitoxantrone, viniblastine or estramustine, provided that such compound has not been used in the previous chemotherapy or cytotoxic chemotherapy regime for hormone refractory prostate cancer.

In another embodiment, the other therapy is chemotherapy that uses a compound that is a taxane, such as paclitaxel or docetaxel, mitoxantrone, viniblastine or estramustine, where such compound has been used in the previous chemotherapy or cytotoxic chemotherapy regime for hormone refractory prostate cancer.

### Other Aspects of the Invention

In an alternative aspect of the invention, the prednisone used in any of the methods, packaged-pharmaceutical-products or uses recited above, is replaced with a corticosteroid. In certain embodiments of this aspect, the corticosteroid is selected from dexamethasone, hydrocortisone or cortisone acetate. In another embodiment of this aspect, the corticosteroid is prednisolone.

In yet another alternative aspect of the invention, the individual is not administered a corticosteroid such as prednisone.

In certain embodiments of the methods, packaged-pharmaceutical-products or uses of such aspects, the administration of a therapeutic amount of satraplatin is single-agent administration, or as single-agent chemotherapy, for treating an individual, or to an individual suffering from metastatic hormone refractory prostrate cancer.

For any of these alternate aspects of the invention, further specific and appropriate embodiments can be envisioned by a person of ordinary skill based on the disclosure herein, including from one or more of the particular embodiments of the inventions listed above, including any combination thereof. By way of non-limiting example, the dose of satraplatin to be orally administered in such alternate aspects can be between about 30 mg/m² and about 140 mg/m², and in particular embodiments a therapeutic amount of antiemetic agent may be administered on the same days as administration of satraplatin.

### Formulations, Dosages and Applications

The compositions of this invention can be formulated and administered to treat individuals in need by any means that produces contact of the active ingredient with the agent's site of action in the body of an individual. They can be administered by any conventional means available for use in conjunction with pharmaceuticals, either as individual therapeutic active ingredients or in a combination of therapeutic active ingredients. They can be administered alone, but are generally administered with a pharmaceutically acceptable diluent, excipient or carrier selected on the basis of the chosen route of administration and standard pharmaceutical practice.

Pharmaceutical compositions for use in accordance with the present invention may be formulated in conventional manner using one or more pharmaceutically acceptable diluents, excipients or carriers. The pharmaceutical compositions of the invention can be formulated for a variety of routes of administration, including systemic and topical or localized administration. Techniques and formulations generally may be found in Remington's Pharmaceutical Sciences, Meade Publishing Co., Easton, Pa. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, capsules, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intrarectally, for example, as a cream or foam. In certain embodiments, the pharmaceutical preparations may be non-pyrogenic, i.e., do not substantially elevate the body temperature of a patient.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the individual being treated, as well as the particular mode of administration. The amount of active ingredient which can be combined with a carrier material to produce a single dosage form will generally be that amount of active ingredient which produces a therapeutic effect when administered as a single or small number of such dosage forms. Generally, out of one hundred percent, this amount will range from about 1 percent to about ninety-nine percent of active ingredient, preferably from about 5, percent to about 70 percent, or in particular embodiments from about 10 percent to about 30 percent.

Methods of preparing these formulations or compositions include the step of bringing into association a compound used in the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product. These formulations may be further prepared shortly before administration of the active ingredient. For example, a formulation may be shaken, diluted or dissolved, a pill divided or crushed, or a syringe filled, often in each case only a few moments before administration to the patient.

Pharmaceutical compositions for use in the invention may be formulated to be suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound used in the present invention as an active ingredient. A compound used in the present invention may also be administered as a bolus, electuary or paste.

In formulating the pharmaceutical compositions for use in the invention in solid dosage forms for oral administration (capsules, tablets, pills, dragees, powders, granules and the like), a compound of the invention as active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, high molecular weight polyethylene glycols, and the like.

Gelatin capsules can contain a compound used in the present invention an as active ingredient, together with powdered carriers, such as lactose, starch, cellulose derivatives, magnesium stearate, stearic acid, and the like. Similar carriers can be used to make compressed tablets. Both tablets and capsules can be manufactured as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract. Solid compositions of a similar type are also employed as fillers in soft and hard-filled gelatin capsules; preferred materials in this connection also include lactose or milk sugar as well as high molecular weight polyethylene glycols. A preferred formulation is a solution or suspension in an oil, for example olive oil, Miglyol, or Capmul, in a soft gelatin capsule. Antioxidants may be added to prevent long-term degradation as appropriate.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor moistened with an inert liquid diluent.

The tablets and other solid dosage forms of the pharmaceutical compositions used in the present invention, such as dragees, capsules, pills and granules, may optionally be scored or prepared with coatings and shells, such as enteric coatings and other coatings well known in the pharmaceutical-formulating art. They may also be formulations so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the pharmaceutical compositions of the invention include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the pharmaceutical compositions for oral administration can also include adjuvants such as welting agents, emulsifying and suspending agents, sweetening, flavoring, coloring, perfuming, and preservative agents.

Suspensions, in addition to the pharmaceutical composition of the present invention, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

For buccal administration the pharmaceutical compositions may take the form of tablets or lozenges formulated in a conventional manner.

For administration by inhalation, the pharmaceutical compositions used in the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the therapeutic agents and a suitable powder base such as lactose or starch.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The pharmaceutical compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions of this invention suitable for parenteral administration comprise one or more compounds used in the invention in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions used in the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These pharmaceutical compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the pharmaceutical compositions. In addition, prolonged absorption of the injectable pharmaceutical form may be brought about by the inclusion of agents that delay absorption such as aluminum monostearate and/or gelatin.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the pharmaceutical compositions used in the invention are formulated into ointments, salves, gels, or creams as generally known in the art. A wash solution can be used locally to treat an injury or inflammation to accelerate healing.

Pharmaceutical compositions for use in the invention may be formulated for rectal administration as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum cavity and release the active inhibitor.

Dosage forms for the topical or transdermal administration of a compound used in this invention include powders, sprays, ointments, pastes, creams, lotions, gels, solutions, patches and inhalants. Such compound may be mixed under sterile conditions with a pharmaceutically acceptable carrier, and with any preservatives, buffers, or propellants which may be required.

The ointments, pastes, creams and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing an inhibitor of the present invention in the proper medium. Absorption enhancers can also be used to increase the flux of the drug across the skin. The rate of such flux can be controlled by either providing a rate controlling membrane or dispersing the compound used in the present invention in a polymer matrix or gel.

Ophthalmic formulations, eye ointments, powders, solutions and the like, are also contemplated as being within the scope of this invention.

The pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. In other embodiments, the pack or dispenser may be further packaged in an outer carton forming one example of a packaged-pharmaceutical-product.

A pharmaceutical composition of the present invention can also be formulated as a sustained and/or timed release formulation. Such sustained and/or timed release formulations may be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions used in the present invention can be used to provide slow or sustained release of one or more of the active ingredients using, for example, hydroxypropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, may be readily selected for use with the pharmaceutical compositions used in the invention. Thus, single unit dosage forms suitable for oral administration, such as, but not limited to, tablets, capsules, gelcaps, caplets, powders, and the like, that are adapted for sustained release are encompassed by the present invention.

Injectable depot forms are made by forming microencapsulated matrices of a compound or drug used in the invention in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The formulations will contain an appropriate amount of the active ingredient or compounds used in the invention. Such amount will depend on a number of factors, including the mode of administration, therapeutic regime or procedure. An appropriate number of amount of the formulation will be administered to the patient, to provide a final dose or amount of active ingredient or compound. Exemplary doses include milligram or microgram amounts of the compounds of the present invention per kilogram of individual or patient weight, e.g., about 1 microgram per kilogram body-weight to about 500 milligrams per kilogram, about 100 micrograms per kilogram to about 50 milligrams per kilogram, or about 1 milligram per kilogram to about 5 milligrams per kilogram.

A person skilled in the art will appreciate that doses can also be calculated on a body surface area (BSA) basis. Such dose rates can be used to calculate the amount of a compound to be used in chemotherapy, such as that set out in the clinical trial described in the exemplification. For example, a person of 70 kg has an approximate body surface area of 1.8 square meter, and doses can be expressed as milligram or microgram amounts of the compound per body surface area of subject or sample, e.g. about 50 micrograms per square meter to about 15 grams per square meter, about 5 milligrams per square meter to about 1.5 grams per square meter, or about 50 milligrams per square meter to about 150 milligrams per square meter.

Alternatively, doses of compounds to be administered to individuals in need thereof, can be expressed as absolute amounts, such as 5 mg prednisone, 1 mg granisetron or 160 mg satraplatin.

### Data Supporting Efficacy

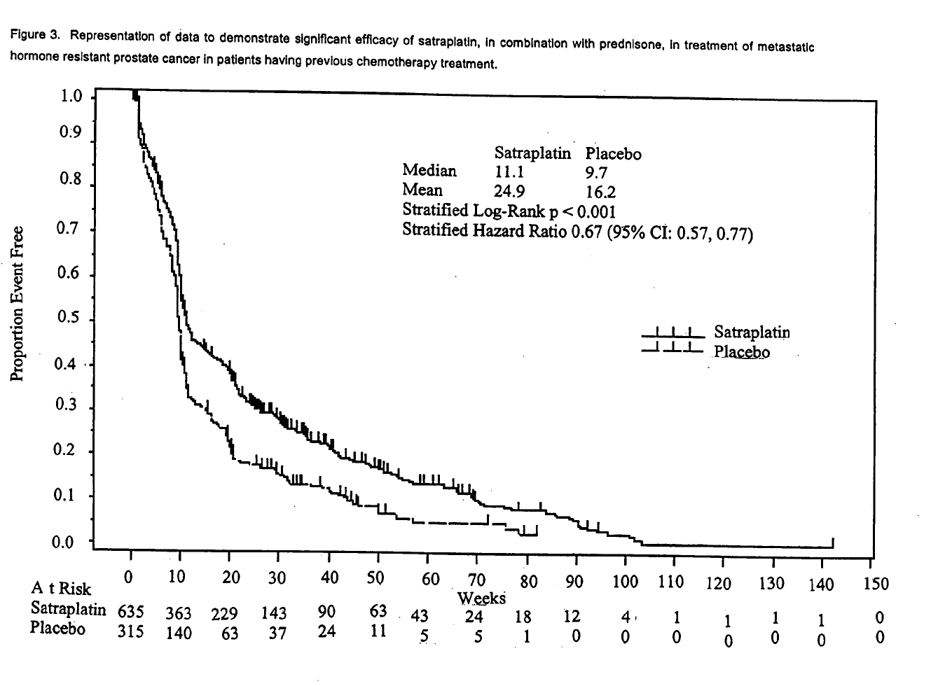

FIG. 3 shows exemplary data from the SPARC trial having a statistically significant difference between and in favour of the satraplatin plus prednisone arm compared to the placebo plus prednisone arm, and the conclusions from the SPARC trial are shown in Exhibit A and Exhibit B. The majority of progression events (70% and 80% of progression event in the satraplatin plus prednisone and placebo plus prednisone arms, respectively) comprised of radiographic progression (37% vs 35%) and pain progression (56% vs 41%).

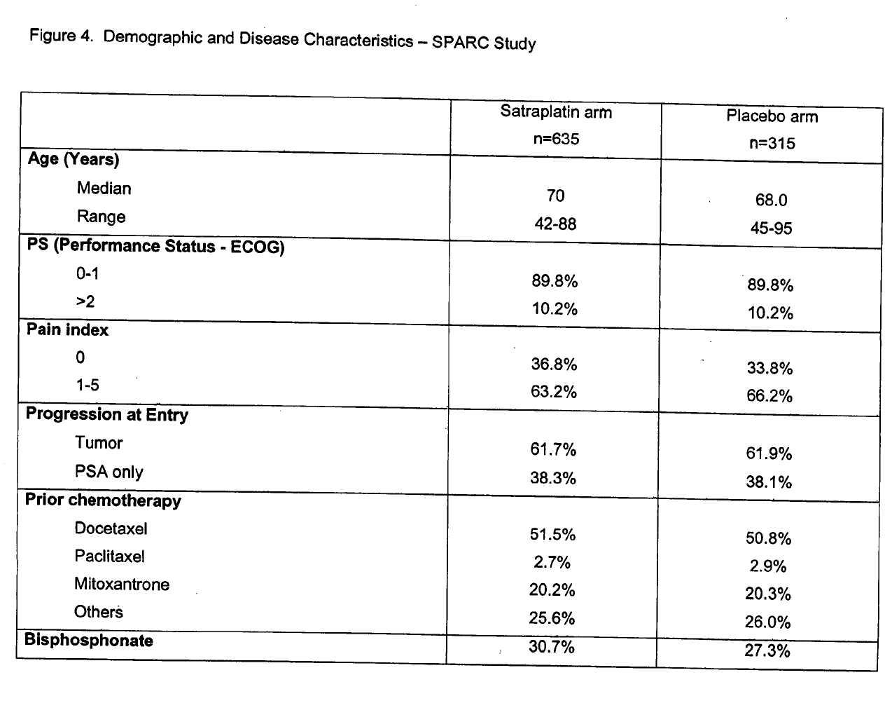

The baseline characteristics of the SPARC trail are summarized in FIG. 4. The two arms show balance of the demographics shown upon entry to the SPARC trail. About half the patients received prior docetaxel (Taxotere®) chemotherapy.

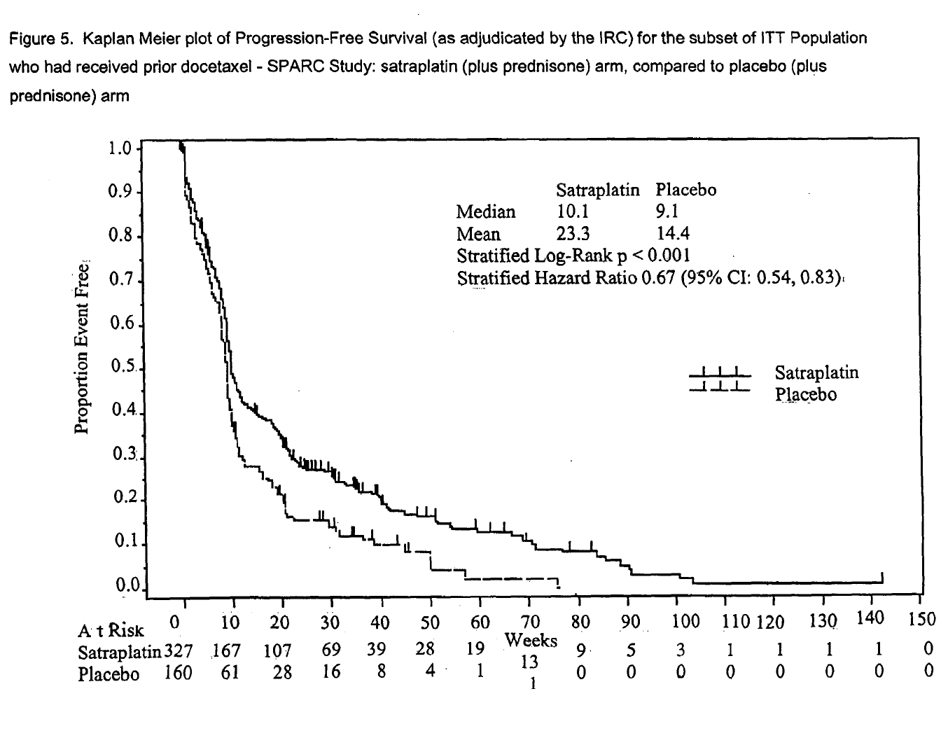

The significant benefit in PFS seen for the satraplatin (plus prednisone) arm, compared to placebo (plus prednisone) arm was maintained irrespective of whether patients had received prior docetaxel (Taxotere®)) chemotherapy (FIG. 5). These data support that satraplatin (plus prednisone) gives a full treatment effect in advanced (metastatic) HRPC patients who have failed prior Taxotere chemotherapy.

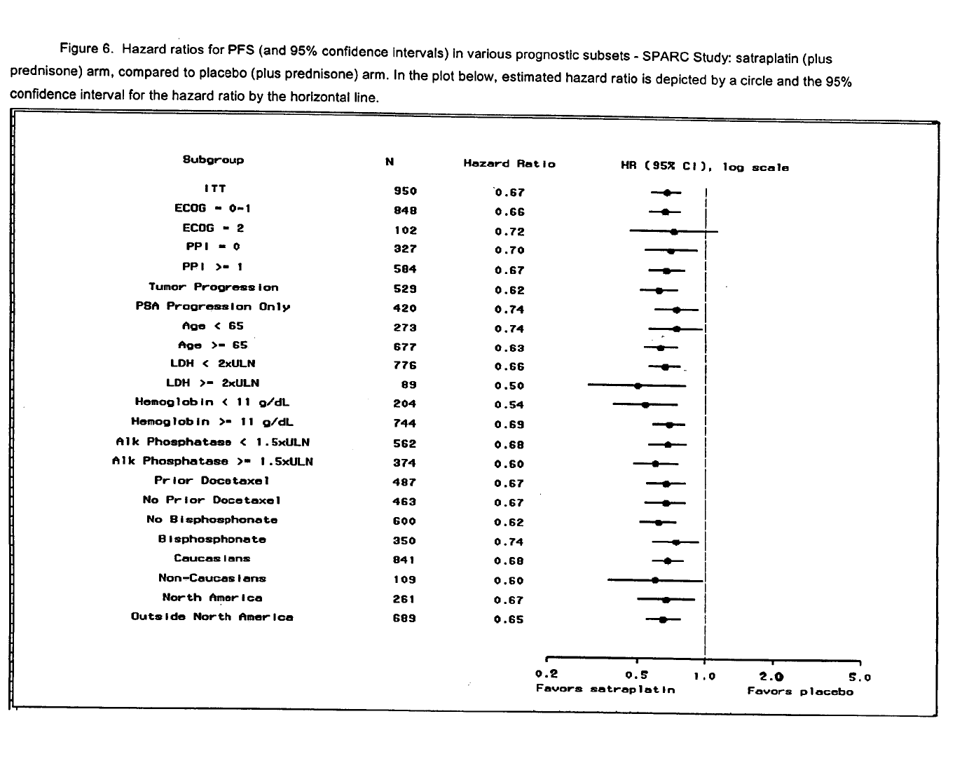

Indeed, the efficacy of the satraplatin (plus prednisone) arm is consistent across patient subsets, showing benefit (as reflected by hazard ratios—“HR”—of less than 1.0) compared to patients on the placebo (plus prednisone) arm, including for those baseline characteristics of patients: highly symptomatic at baseline (pain), PSA progression only, age, haemoglobin and alkaline phosphatase levels, prior docetaxel and biphosphonate use (FIG. 6).

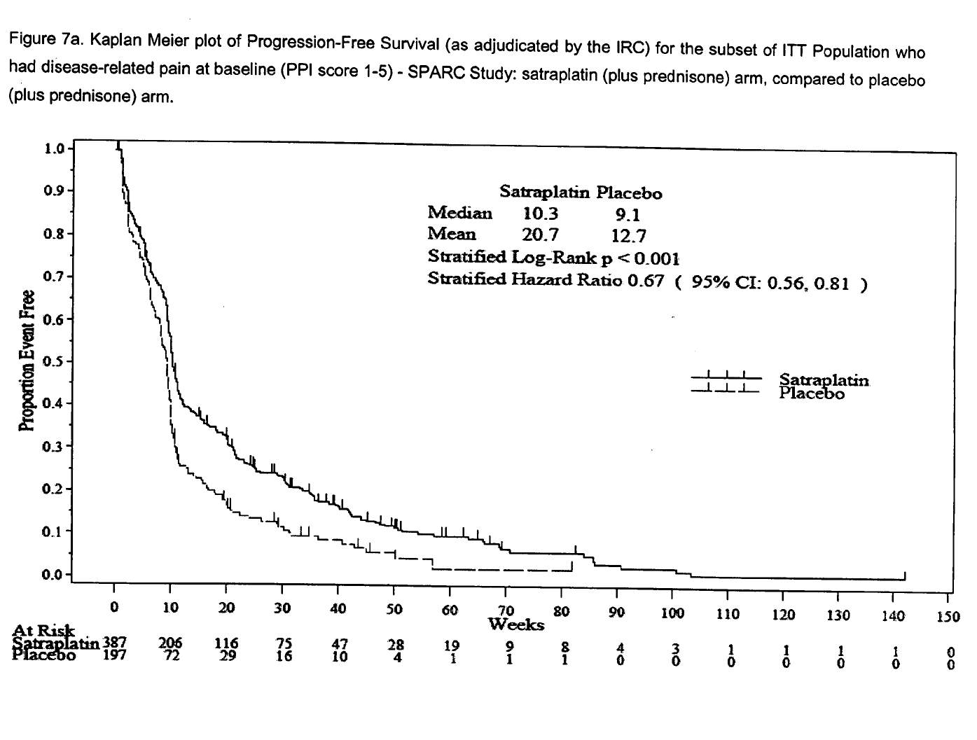

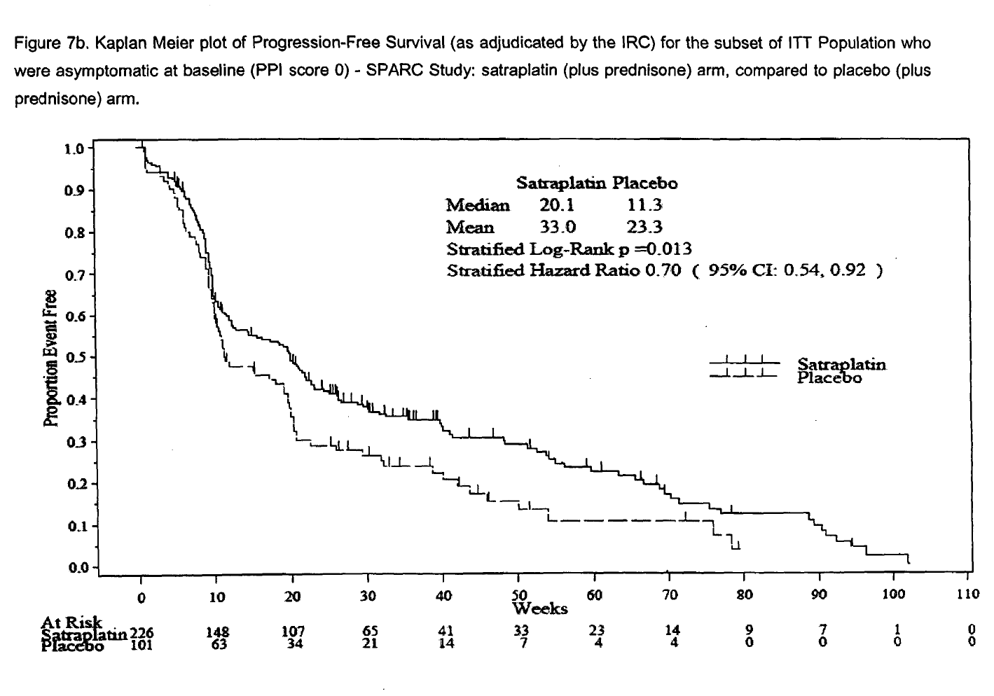

The SPARC study included both symptomatic and asymptomatic patients, as defined by baseline PPI score. For PFS, treatment effects of similar magnitude, favoring the satraplatin (plus prednisone) arm were obtained for the ITT population (HR=0.67, 95% Cl: 0.57, 0.77) and the subsets of the ITT population with disease-related pain at baseline (PPI score 1-5; HR=0.67, 95% Cl: 0.56, 0.81) and no pain at baseline (PPI score 0; HR=0.70, 95% Cl: 0.54, 0.92). Kaplan Meier plots for PFS for these ITT subsets are shown in FIGS. 7a and 7b. Indeed, a benefit in favour of the satraplatin (plus prednisone) arm was seen in the median progression free survival for asymptomatic patients (PPI=0) compared to symptomatic patients (PPI of 1 to 5). Asymptomatic patients on the satraplatin (plus prednisone) arm showed a median PFS of 20.1 weeks compared to 11.3 weeks for patients on the placebo (plus prednisone) arm (a difference of 8.8 weeks), compared to 10.3 to 9.1 weeks for symptomatic patients (a difference of 1.2 weeks).

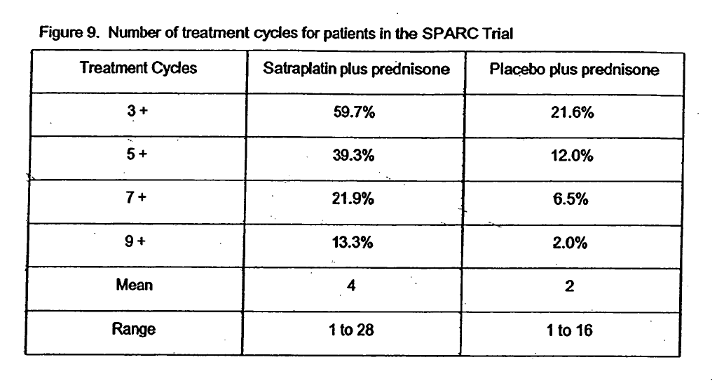

The extension of progression free survival shown by the patients on the satraplatin (plus prednisone) arm is associated with an increase in the increased number of cycles of treatment such patients were administered, compared to the number of cycles administered to patients on the placebo (plus prednisone) arm (FIG. 9).

In the SPARC study, there was a 34% lower risk of pain progression for patients on the satraplatin (plus prednisone) arm compared to patients on the placebo (plus prednisone) arm (HR=0.66, 95% Cl: 0.50-0.83). The mean Time to Pain Progression (TPP) for patients on the satraplatin (plus prednisone) arm was 53.4 weeks compared to 36.6 weeks for those on the placebo (plus prednisone) arm (p<0.001). Furthermore, in patients that were symptomatic for pain at baseline, 24% of patients on the satraplatin plus prednisone arm experienced a Pain Response (reduction in pain) compared to 14% on the placebo plus prednisone arm (p=0.0047), with a duration of such Pain Response of 40.1 vs 24.1 weeks respectively.

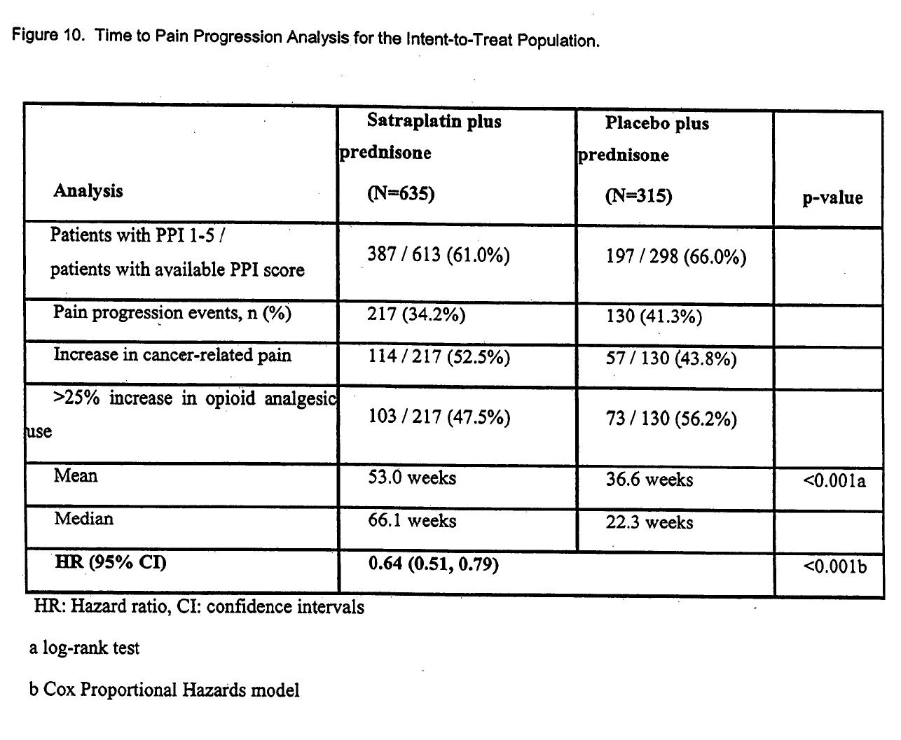

For the ITT population, FIG. 10 shows additional analyses of TPP, pain progression or opioid-use data, displaying benefits in favour of the satraplatin plus prednisone arm.

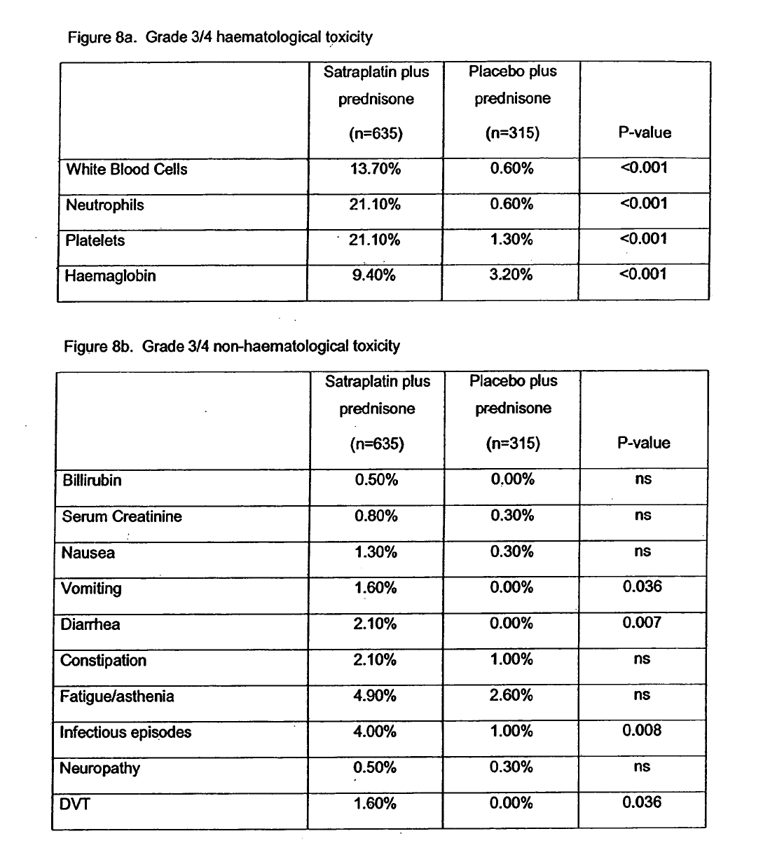

Therapy with satraplatin plus prednisone was well tolerated, with the most common treatment-related adverse events being those haemotological events associated with myelosupression (FIG. 8a). Other (non-haematological Grade 3 or Grade 4 toxicities are listed in FIG. 8b. Importantly, the incidence of any serious non-haematological toxicity was less than 5% of patients treated with satraplatin plus prednisone. Of note is that, unlike treatment with other platinum compounds, satraplatin plus prednisone did not show a significantly higher incidence of neuropathy and renal events compared to placebo plus prednisone.

### ADDITIONAL REFERENCES

Bubley G J, Carducci M, Dahut W, et al. Eligibility and Response Guidelines for Phase II Clinical trials in Androgen-Independent Prostate Cancer: Recommendations From the Prostate-Specific Antigen Working Group. J Clin Oncol 1999; 17:3461-7.
Dagher R et al Docetaxel in Combination with Prednisone for the Treatment of Androgen-independent Hormone-Refractory Prostate Cancer. Clin Cancer Res 2004, 10: 8147-8151.
Dawson N A. Treatment of progressive metastatic prostate cancer. Oncology 1993; 7:17-27.
Eisenberger M A, Simon R, O'Dwyer P J, Wittes R E, Friedman M A. A reevaluation of non-hormonal cytotoxic chemotherapy in the treatment of prostate cancer. J Clin Oncol 1985; 3:827-41.
Eisenberger M A, Nelson W G. How much can we rely on the level of prostate-specific antigen as an end point for evaluation of clinical trials? A word of caution. J Nat Can Inst 1996; 88(12):779-81.
Eisenberger M, De Wit R, Berry W, et al. A multicenter phase III comparison of docetaxel (D)+prednisone and mitoxantrone (MTZ)+P in patients with hormone-refractory prostate cancer (HRPC). Proc Am Soc Clin Oncol 2004; 23:abstract #4.
Hudes G, Nathan F, Khater C, et al. Phase II trial of 96-hour paclitaxel plus oral estramustine phosphate in metastatic hormone-refractory prostate cancer. J Clin Oncol 1997; 15:3156-63.
Hussain M, Petrylak D, Fisher E, Tangen C, Crawford D. Docetaxel (Taxotere) and estramustine versus mitoxantrone and prednisone for hormone-refractory prostate cancer: scientific basis and design of Southwest Oncology Group Study 9916. Semin Oncol 1999; 26 (5Suppl 17):55-60.
Kelly W, Slovin S. Chemotherapy for Androgen-independent Prostate Cancer: Myth or Reality. Current Oncol Rep 2000; 2:394-401.
Loehrer P J, Einhorn L H. Cisplatin. Ann Int Med 1984; 100:704-13.
Merrin C E. Treatment of genitourinary tumors with cis-dichloroammineplatinum (II): experience in 250 patients. Cancer Treat Rep 1979; 63:1579-84.
Miller J I, Ahmann F R, Drach G W, Emerson S S, Bottaccini M R. The clinical usefulness of serum prostate specific antigen after hormonal therapy of metastatic prostate cancer. J Urol 1992; 147:956-61.
Moore M R, B T M, deSimone P, Birch R, Irwin L. Phase II evaluation of weekly cisplatin in metastatic hormone-resistant prostate cancer: A Southeastern Cancer Study Group trial. Cancer Treat Rep 1986; 70:541-2.
Petrylak D P, Macarthur R, O'Connor J, et al. Phase I/II studies of docetaxel (Taxotere) combined with estramustine in men with hormone-refractory prostate cancer. Semin Oncol 1999; 26(5 Suppl 17):28-33.
Petrylak D, Tangen C, Hussain M, et al. SWOG 99-16: Randomized phase III trial of docetaxel (D)/estramustine (E) versus mitoxantrone (M)/prednisone(p) in men with androgen-independent prostate cancer (AIPCA). Proc Am Soc Clin Oncol 2004; 23:abstract #3.
Pienta K J, Redman B, Hussain M, et al. Phase II evaluation of oral estramustine and oral etoposide in hormone-refractory adenocarcinoma of the prostate. J Clin Oncol 1994; 12:2005-21.
Prestayko A, D'Aoust J, Issell B, Crooke S. Cisplatin (cis-diaminedichloroplatinum II). Cancer Treat Rev 1979; 6:17-39.
Qazi R, Khandekar J-. Phase II study of cisplatin for metastatic prostatic cancer. Am J Oncol 1983; 6:203-5.
Raynaud et al. 1996 Cancer Chemother Phamacol 38: 155-162
Rossof A H, Talley R W, Stephens R, et al. Phase II evaluation of cisdichlorodiammineplatinum (II) in advanced malignancies of the genitourinary and gynecologic organs: a Southwest Oncology Group study. Cancer Treat Rep 1979; 63:1557-64.
Rozencweig M, Von Hoff D D, Slavik M, Muggia F M. Cis-diaminedichloroplatinum (II) a new anticancer drug. Ann Int Med 1977; 86:803-12.
Soloway M, S B, Brady M, et al. A comparison of estramustine phosphate versus cisplatinum alone versus estramustine phosphate plus cisplatinum in patients with advanced hormone refractory prostate cancer who had extensive irradiation to the pelvis or lumbosacral area. J Urol 1983; 129:56-61.
Sternberg C. Hormone refractory metastatic prostate cancer. Ann Oncol 1992; 3(5):331-5.
Tannock I F, Osoba D, Stockier M R, et al. Chemotherapy with mitoxantrone plus prednisone or prednisone alone for symptomatic hormone-resistant prostate cancer: A Canadian randomized trial with palliative end points. J Clin Oncol 1996; 14(6):1756-64.
Yagoda A. Phase II trials with cis-dichlorodiammineplatinum (II) in the treatment of urothelial cancer. Cancer Treat Rep 1979 (1); 63:1565-72.
Yagoda A, Watson R, Natale R. A critical analysis of response criteria in patients with prostate cancer treated with cis-diamminedichlororide platinum (II). Cancer 1979 (II); 44:1553-62.
Yagoda A, Petrylak D P. Cytotoxic chemotherapy for advanced hormone-resistant prostate cancer. Cancer 1993; 71 (Supplement): 1098-109.

### EQUIVALENTS

Those skilled in the art will recognize, or be able to ascertain using no more than routine experimentation, many equivalents to the specific embodiments of the invention described herein. Such equivalents are intended to be encompassed by the following claims.

All of the above-cited references and publications are hereby incorporated by reference.

1. A method of treating an individual suffering from metastatic hormone refractory prostate cancer comprising administration of a therapeutically effective amount of satraplatin to said individual, wherein:
(a) said individual was treated with previous chemotherapy for metastatic hormone refractory prostate cancer; and
(b) said method further comprises the administration of a corticosteroid to said individual in combination with said administration of satraplatin.

2. A packaged-pharmaceutical-product comprising a pharmaceutical composition that includes satraplatin, wherein said packaged-pharmaceutical-product further comprises instructions to conduct administration of a therapeutically effective amount of said satraplatin included in said pharmaceutical composition to an individual suffering from metastatic hormone refractory prostate cancer, wherein said instructions further include:
(a) an instruction to conduct said administration of satraplatin to an individual who was treated with previous chemotherapy for metastatic hormone refractory prostate cancer; and
(b) an instruction to conduct said administration of satraplatin in combination with administration of a corticosteroid.

3. The packaged-pharmaceutical-product of claim 2, further comprising a second pharmaceutical composition that includes a corticosteroid.

4. (canceled)

5. The method according to claim 1, wherein said individual has a diagnosis of Stage D2 adenocarcinoma of the prostate that is unresponsive to hormone therapy.

6. The method according to claim 1, wherein said individual has failed treatment with said previous chemotherapy.

7. The method according to claim 1, wherein said individual has taken a chemotherapy holiday from said previous chemotherapy.

8. The method method according to claim 1, wherein said chemotherapy was a cytotoxic chemotherapy regime.

9. The method according to claim 1, wherein said individual has suffered disease progression or PSA progression after a minimum of two courses of one prior cytotoxic chemotherapy regime for metastatic hormone refractory prostate cancer.

10. The method according to claim 8, wherein said chemotherapy used a compound selected from mitoxantrone, viniblastine, estramustine and a taxane.

11. The method according to claim 9, wherein said cytotoxic chemotherapy regime used a compound selected from mitoxantrone, viniblastine, estramustine and a taxane.

12. The method according to claim 1, wherein said chemotherapy or cytotoxic chemotherapy regime used a taxane.

13. The method, packaged pharmaceutical product or use of according to claim 12, wherein said taxane was paclitaxel or docetaxel.

14. The method according to claim 13, wherein said taxane was docetaxel.

15. The method according to claim 1, wherein said individual is administered satraplatin orally at a dose of between about 30 mg/m² and about 140 mg/m² per day over between 3 and 7 days.

16. The method according to claim 15, wherein said individual is administered satraplatin orally at a dose of between about 40 mg/m² and about 100 mg/m² per day over between 3 and 7 days.

17. The method according to claim 16, wherein said individual is administered satraplatin orally at a dose of between about 50 mg/m² and about 90 mg/m² per day over between 3 and 7 days.

18. The method according to claim 15, wherein said individual is administered satraplatin orally at a dose of about 40 mg/m² per day over between 3 and 7 days.

19. The method according to claim 16, wherein said individual is administered satraplatin orally at a dose of about 60 mg/m² per day over between 3 and 7 days.

20. The method according to claim claim 17, wherein said individual is administered satraplatin orally at a dose of about 80 mg/m² per day over between 3 and 7 days.

21. The method according to claim 1, wherein said individual is administered satraplatin daily for about five consecutive days, with the cycle repeated about every 35 days.

22. The method according to claim 1, wherein no food is taken by said individual for at least about one hour before, and for at least about 2 hours after said administration of satraplatin.

23. The method according to claim 1, wherein said administration of satraplatin is to said individual on an empty stomach.

24. The method according to claim 1, wherein said individual is administered a corticosteroid orally with an amount of between 2 mg and 10 mg twice per day.

25. The method according to claim 24, wherein said individual is administered a corticosteroid orally at an amount of 5 mg twice per day.

26. The method according to claim 1, wherein said individual is administered a corticosteroid orally about one hour prior to administration of satraplatin orally and about eight hours after administration of satraplatin orally.

27. The method according to claim 25, wherein said individual is administered a corticosteroid in the morning and the evening on those days of a cycle when satraplatin is not administered.

28. The method according to claim 1, wherein said individual is further administered an antiemetic agent on the same day as said administration of satraplatin.

29. The method according to claim 28, wherein said antiemetic agent is administered about one hour prior to administration of satraplatin orally and about eight hours after administration of satraplatin orally.

30. The method according to claim 28, wherein said antiemetic agent is a 5-HT3 blocker or inhibitor.

31. The method according to claim 30, wherein said 5-HT3 blocker or inhibitor is granisetron.

32. The method according to claim 31, wherein said granisetron is administered orally with an amount of between 0.2 mg and 5 mg.

33. The method according to claim 32, wherein said granisetron is administered orally with an amount of 1 mg.

34. The method according to claim 1, comprising the steps of:
(a) administering to said individual, on each of days 1 to 5, a corticosteroid (5 mg) and an antiemetic agent (1 mg) orally, followed after about 1 hour by the administration of satraplatin orally at a dose of about 80 mg/m², followed after about 8 hours by the administration of a corticosteroid (5 mg) and an antiemetic agent (1 mg) orally;
(b) administering to said individual, on each of days 6 to 35, a corticosteroid (5 mg) twice daily in the morning and evening; and
(c) repeating (a) and (b) at least one time.

35. The packaged-pharmaceutical-product according to claim 2, wherein the instructions included in said packaged-pharmaceutical-product comprise instructions to conduct the steps of:
(a) administering to said individual, on each of days 1 to 5, a corticosteroid (5 mg) and an antiemetic agent (1 mg) orally, followed after about 1 hour by the administration of satraplatin orally at a dose of about 80 mg/m², followed after about 8 hours by the administration of a corticosteroid (5 mg) and antiemetic agent (1 mg) orally;
(b) administering to said individual, on each of days 6 to 35, a corticosteroid (5 mg) twice daily in the morning and evening; and
(c) repeating (a) and (b) at least one time.

36. (canceled)

37. The method according to claim 34, wherein said administration of satraplatin is to said individual on an empty stomach.

38. The method according to claim 34, wherein said administration of satraplatin results in an elongation of the time to disease progression.

39. The method according to claim 34, wherein said administration of satraplatin results in an elongation of the progression-free survival.

40. The method according to claim 34, wherein said administration of satraplatin results in an elongation of the overall survival.

41. The method according to claim 34, wherein said corticosteroid is prednisone.
